(19) United States
(12) Patent Application Publication
(10) Pub. No.: US 2026/0279546 A1
(43) Pub. Date: Sep. 17, 2026

(54) MACHINE-LEARNING INGESTION LAYER FOR FACIAL LANDMARK REFINEMENT AND MEASUREMENT ACCURACY

(71) Applicant: SITREP HEALTH, LLC, Livermore, CA (US)

(72) Inventors: Ryan WILKERSON, Livermore, CA (US); lan SIDDIQI, Valley Village, CA (US)

(73) Assignee: SITREP HEALTH, LLC, Livermore, CA (US)

(21) Appl. No.: 19/265,975

(22) Filed: Jul. 10, 2025

Related U.S. Application Data
(60) Provisional application No. 63/773,281, filed on Mar. 17, 2025.

(51) Int. Cl. G16H 30/40; G06V 20/40; G06V 40/16; G16H 20/00

(52) U.S. Cl. CPC G16H 30/40; G06V 20/46; G06V 40/171; G16H 20/00

(57) ABSTRACT

Disclosed herein are systems and methods for enhancing facial landmark accuracy via machine learning-based ingestion layer. The method includes: performing a facial scan and generating a plurality of video frames from the captured image; extracting facial landmark estimates corresponding to the face of the user using a pre-trained machine learning (ML) model configured to identify anatomical features of the face; generating refined facial landmark estimates by inputting the extracted facial landmark estimates into a ML ingestion layer configured to perform at least one of: filtering noise, correcting drift, or improving reliability; and generating prescription-related measurements for the user based on the refined facial landmark estimates, wherein the prescription-related measurements comprises at least one of a temple to temple width, temple arm length, or a nose bridge width.

### CROSS REFERENCE TO RELATED APPLICATIONS

This application claims priority to U.S. Patent Provisional Application No. 63/773,281, filed Mar. 17, 2025, the entire contents of which are hereby incorporated by reference.

### FIELD OF TECHNOLOGY

The present disclosure relates to the field of machine learning, and, more specifically, to systems and methods for determining facial measurements as part of an eyewear recommendation process.

### BACKGROUND

Workers in industries such as construction, manufacturing, mining, carpentry, auto repair, electrical work, plumbing, and welding face daily hazards that pose significant risks to their vision. Protective eyewear is a critical component of workplace safety, shielding workers from potential injuries caused by flying debris, chemical splashes, sparks, and excessive light exposure. Despite its importance, the process of selecting properly fitting protective eyewear remains outdated, often relying on manual measurements and subjective assessments by opticians. Ill-fitting eyewear can lead to discomfort, prescription misalignment, and reduced effectiveness in protecting against occupational hazards, increasing the likelihood of eye strain, fatigue, and workplace accidents. Given the stringent safety standards in these industries, ensuring precise, well-fitted eyewear is essential not only for compliance but also for enhancing worker safety, productivity, and overall well-being. As workplace environments evolve with technological advancements, the demand for more accurate, efficient, and AI-powered eyewear fitting solutions continues to grow, making innovation in this field both necessary and timely.

In systems employing facial landmark detection for generating facial fit measurements, the detected landmark coordinates are typically expressed in pixel-space (i.e., as two-dimensional locations or three-dimensional locations within the coordinate system of a digital image). However, pixel-space coordinates are inherently dependent on the external factors such as resolution, focal length, sensor size, and geometric positioning of the camera relative to the subject, and thus lack any intrinsic real-world dimensionality. Consequently, direct use of pixel-space landmark data in applications requiring precise real-world fit measurements (e.g., in eyewear fitting, respiratory mask sealing, or helmet sizing) results in scale ambiguity and geometrical distortion. To resolve this, a reliable and consistent transformation from pixel-space to real-world units (e.g., millimeters) must be performed.

### SUMMARY

To address inefficiencies in current eyewear fitting processes, the present disclosure describes the use of machine learning (ML) models to generate real-world, landmark-based facial measurements relevant to eyewear prescription. The system captures hundreds of facial frames using an interactive video scan, with a real-time face detection ML model operating at up to 240 frames per second to detect facial landmark estimates. However, these raw landmark detections are often noisy and unstable, making them unreliable for precise measurements such as interpupillary distance (IPD), temple width, and iris diameter. Sources of instability may include detection noise, temporal drift, and inconsistent scaling factors, all of which impact measurement accuracy.

To improve reliability, the present disclosure introduces an ML ingestion layer between the raw landmark output and the real-time facial measurement engine. This layer processes landmark data on a per-frame basis using over 10,000 manually labeled facial scan sequences for supervision. Techniques such as temporal analysis, rolling averages, and Kalman filtering are employed to reduce noise, correct drift, and identify stable anatomical features, including pupil centers, iris edges, nose bridge, and facial width. Landmark sets are evaluated for stability and anatomical accuracy, with low-confidence data discarded. The output is a corrected and stabilized landmark set, suitable for pixel-to-millimeter conversion to support accurate, real-time facial measurements.

In this way, the machine learning (ML) ingestion layer improves point selection accuracy, thereby enhancing the reliability of downstream facial measurements. By further improving landmark stability, the system ensures consistent scaling factors and increased precision in subsequent measurement computations. Additionally, by applying calibration methods to the stabilized landmark data, the system maps two-dimensional pixel-space coordinates or three-dimensional spatial positions in a real-world coordinate system. This enables accurate calculation of inter-landmark distances and anatomical curvatures, ensuring that the resulting facial fit measurements correspond to actual physical dimensions. Collectively, these improvements address the technical challenge of converting inherently dimensionless, image-based observations into consistent, reliable, and actionable biometric data for real-world fit assessment applications.

In one exemplary aspect, a system is provided for enhancing facial landmark accuracy, the system including: an image sensor configured to perform a facial scan by capturing an image of a face of a user and generate a plurality of video frames from the captured image; and a processor coupled to the image sensor, the processor being configured to: extract, from each of the plurality of video frames, facial landmark estimates corresponding to the face of the user using a pre-trained machine learning (ML) model configured to identify anatomical features of the face, wherein the extracted facial landmark estimates comprise at least one of a pupil estimate, an iris edge estimates, a 68-point model, or temple-to-temple width; generate refined facial landmark estimates by inputting the extracted facial landmark estimates into a ML ingestion layer configured to perform at least one of: filtering noise, correcting drift, or improving reliability; and generate prescription-related measurements for the user based on the refined facial landmark estimates, wherein the prescription-related measurements comprises at least one of a temple to temple width, temple arm length, or a nose bridge width.

In some aspects, the techniques described herein relate to a system, wherein the image sensor is configured to: generate the plurality of video frames in pixel space, wherein the processor is further configured to: generate the prescription-related measurements based further on the refined facial landmark estimates in real-world measurements by applying a scaling factor to the extracted facial landmark estimates or to the refined facial landmark estimates.

In some aspects, the techniques described herein relate to a system, the processor is further configured to: determine the scaling factor by (W/iris_width_pixels)+(WIPD/pupil_distance_pixel)/2, wherein W corresponds to an iris width constant and WIPD corresponds to an IPD distance.

In some aspects, the techniques described herein relate to a system, the processor is further configured to improve the reliability of the extracted facial landmark estimates by: tracking the stability of a plurality of the extracted facial landmark estimates, and discarding the extracted facial landmark estimates that have a stability that does not conform to a predetermined stability threshold.

In some aspects, the techniques described herein relate to a system, wherein the machine learning model is trained using a supervised learning approach on a training dataset comprising facial images annotated with ground truth anatomical features.

In some aspects, the techniques described herein relate to a system, wherein the training dataset comprises at least one of: facial images annotated with a distance between an outer or inner corners of both eyes; facial images annotated with left and right boundaries of each iris; facial images annotated with a pupil center for each eye; facial images annotated with landmarks at a widest visible part of the face; facial images annotated with a nasion point or a bottom of a nose bridge; and facial images annotated with a mid-point of an ear to an outer corner of the eye.

According to one aspect of the disclosure, a method is provided for enhancing facial landmark accuracy. The method comprising: performing a facial scan by capturing an image of a face of a user and generating a plurality of video frames from the captured image; extracting, from each of the plurality of video frames, facial landmark estimates corresponding to the face of the user using a pre-trained machine learning (ML) model configured to identify anatomical features of the face, wherein the extracted facial landmark estimates comprise at least one of a pupil estimate, an iris edge estimates, a 68-point model, or temple-to-temple width; generating refined facial landmark estimates by inputting the extracted facial landmark estimates into a ML ingestion layer configured to perform at least one of: filtering noise, correcting drift, or improving reliability; and generating prescription-related measurements for the user based on the refined facial landmark estimates, wherein the prescription-related measurements comprises at least one of a temple to temple width, temple arm length, or a nose bridge width.

According to one aspect of the disclosure, a computer program product embodied in a non-transitory computer-readable storage medium storing instructions for enhancing facial landmark accuracy, which when executed by at least one processor, causes the at least one processor to: perform a facial scan by capturing an image of a face of a user and generate a plurality of video frames from the captured image; extract, from each of the plurality of video frames, facial landmark estimates corresponding to the face of the user using a pre-trained machine learning (ML) model configured to identify anatomical features of the face, wherein the extracted facial landmark estimates comprise at least one of a pupil estimate, an iris edge estimates, a 68-point model, or temple-to-temple width; generate refined facial landmark estimates by inputting the extracted facial landmark estimates into a ML ingestion layer configured to perform at least one of: filtering noise, correcting drift, or improving reliability; and generate prescription-related measurements for the user based on the refined facial landmark estimates, wherein the prescription-related measurements comprises at least one of a temple to temple width, temple arm length, or a nose bridge width.

The above simplified summary of example aspects serves to provide a basic understanding of the present disclosure. This summary is not an extensive overview of all contemplated aspects and is intended to neither identify key or critical elements of all aspects nor delineate the scope of any or all aspects of the present disclosure. Its sole purpose is to present one or more aspects in a simplified form as a prelude to the more detailed description of the disclosure that follows. To the accomplishment of the foregoing, the one or more aspects of the present disclosure include the features described and exemplarily pointed out in the claims.

### BRIEF DESCRIPTION OF THE DRAWINGS

The accompanying drawings, which are incorporated into and constitute a part of this specification, illustrate one or more example aspects of the present disclosure and, together with the detailed description, serve to explain their principles and implementations.

FIG. 1 is a block diagram illustrating a system for a measurement process, a calibration process, and an eyewear recommendation process according to aspects of the present disclosures.

FIG. 2 is a block diagram illustrating a system for determining prescription-related facial measurements for fitting eyewear according to aspects of the present disclosure.

FIG. 3 is a block diagram illustrating a system for preparing a machine learning model (MLM) to extract a set of facial landmark estimates and to select pairs of facial landmark estimates according to aspects of the present disclosure.

FIG. 4 is an example flowchart for calculating distances of facial landmark estimates for each user according to aspects of the present disclosure.

FIG. 5 is a block diagram illustrating a facial landmark mapping system for accurately detecting facial landmark estimates according to aspects of the present disclosure.

FIG. 6 is an example method for enhancing facial landmark accuracy according to an aspect of the present disclosure.

FIG. 7 presents an example of a general-purpose computer system on which aspects of the present disclosure can be implemented.

Like reference numbers and designations in the various drawings indicate like elements.

### DETAILED DESCRIPTION

Exemplary aspects are described herein in the context of a system, method, and computer program product for enhancing facial landmark accuracy via machine learning-based ingestion layer. Those of ordinary skill in the art will realize that the following description is illustrative only and is not intended to be in any way limiting. Other aspects will readily suggest themselves to those skilled in the art having the benefit of this disclosure. Reference will now be made in detail to implementations of the example aspects as illustrated in the accompanying drawings. The same reference indicators will be used to the extent possible throughout the drawings and the following description to refer to the same or like items.

Filling an eyeglass prescription involves a multi-step process that ensures users (e.g., patients) receive corrective lenses tailored to their specific vision needs. An eyeglass prescription is issued by an eyewear prescriber, such as an optometrist, following a comprehensive eye examination. This prescription includes the necessary parameters required to construct and dispense lenses that correct the user's vision. Once the prescription is provided, the user is referred to an optician for frame selection, which is a crucial step in ensuring proper fit and comfort. Traditionally, opticians manually assess three key measurements: frame lens width, nose bridge width, and the overall length of the frame arm. However, rather than relying solely on precise measurements, many opticians conduct only a brief visual assessment and make frame recommendations based on personal judgment.

This traditional fitting process is often inefficient, time-consuming, and prone to inaccuracies, leading to frequent frame returns and dissatisfied customers. Poorly fitting eyewear can result in discomfort, misalignment of the prescription, and aesthetic concerns. Additionally, ill-fitting frames can pose safety hazards, particularly in workplaces where protective eyewear is necessary. Risks include eye strain, fatigue, reduced situational awareness, and persistent headaches, all of which can negatively impact productivity and well-being. In industries such as construction, manufacturing, and auto repair, improperly fitted eyewear fails to shield against light exposure, dust, and debris, increasing the likelihood of workplace accidents and associated compensation claims. These inefficiencies highlight the need for a more precise, data-driven approach to eyewear prescription fulfillment, ensuring a better fit, enhanced comfort, and improved safety for users.

Data-driven recommendations and ML models eliminate guesswork, enabling accurate frame selections tailored to each user's face. This approach enhances efficiency, minimizes fitting errors, and significantly reduces high return rates associated with poorly fitting eyewear. In addition, the present disclosure improves the overall user experience, providing a seamless, accurate, and personalized eyewear fitting process.

The present disclosure introduces a ML ingestion layer between the raw landmark output and the real-time facial measurement engine. This layer processes each video frame and applies techniques such as temporal analysis, rolling averages and/or Kalman filtering to reduce noise, correct drift, and/or stabilize key anatomical features (e.g., pupil centers, iris edges, nose bridge, and facial width). Any unstable or low-confidence landmarks are discarded. The result is a corrected and stable set of facial landmark estimates, suitable for accurate pixel-to-millimeter conversion in real-time measurements for downstream processing.

Turning now to the figures, example aspects are depicted with reference to one or more components described herein, where components in dashed lines may be optional.

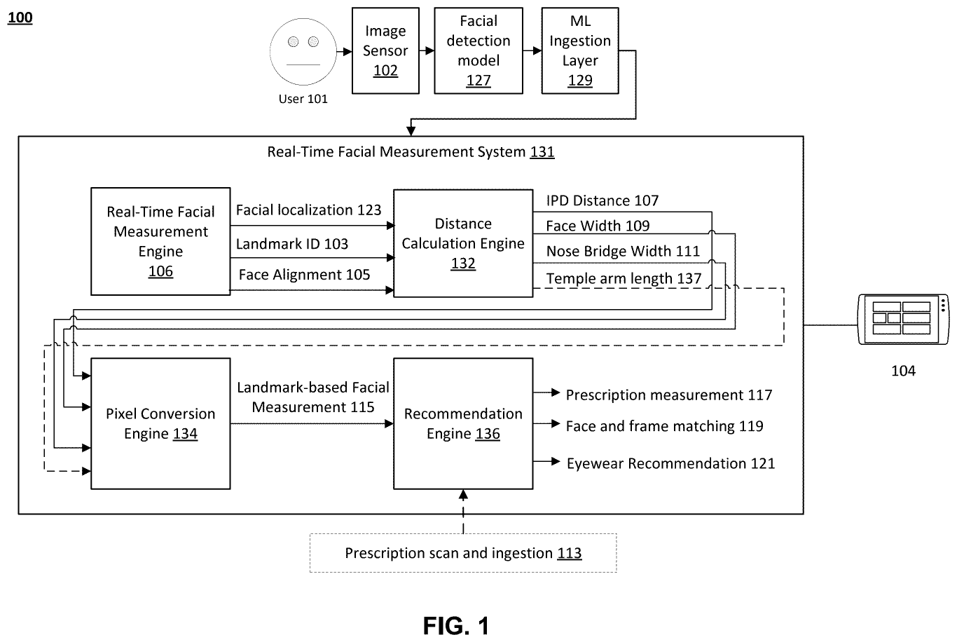

FIG. 1 is a block diagram illustrating a real-time facial measurement system 100 for implementing a measurement process and an eyewear recommendation process according to aspects of the present disclosure. In various implementations, the method of the system 100 is performed by a device with one or more processors and non-transitory memory that performs predictions. In some implementations, the method is performed by processing logic, including hardware, firmware, software, or a combination thereof.

An example of the system 100 may include an image sensor 102, a facial detection model 127, a ML ingestion layer 129, a real-time facial measurement system 131 including at least a real-time facial measurement engine 106, a distance calculation engine 132, a pixel conversion engine 134, and a recommendation engine 136, and a computing device 104 for displaying outputs. At a high level, the system 100 is configured to collect hundreds of facial data points during scanning, which are analyzed using generative artificial intelligence (AI) to determine the best-fitting eyewear frame(s) for a user. Unlike fixed-measurement methods, the system 131 dynamically adapts to each user's unique facial structure. For example, the system 131 may convert pixel data into precise measurements, which are then matched against frame specifications. The system 131 further personalizes recommendations by factoring in profile data such as age, sex, ethnicity, and face shape. In some aspects, recommended frames are ranked by fit accuracy, and the platform continuously improves through self-learning, real-time data processing, and predictive modeling for future styles.

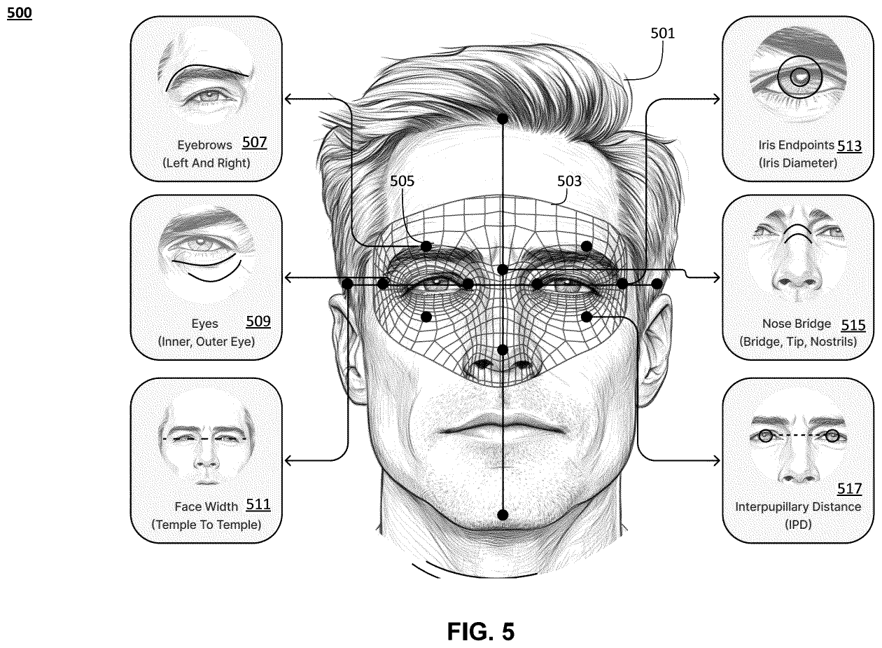

First, the image sensor 102 may be configured to perform a facial scan by capturing an image of a face of a user and generate a plurality of video frames from the captured image for performing continuous facial landmark tracking. A facial detection model 127 then processes each frame of the video data by performing face detection to identify a region of interest containing a user's face. This region is then processed by the facial detection model 127 to output a set of hundreds of facial landmark estimates (e.g., 468 facial landmark points) for the user, each comprising normalized x, y, and z coordinates, where x and y represent 2D positions relative to the frame dimensions and z indicates relative depth. These facial landmark estimates correspond to measurements of anatomically significant facial points including the eyes, nose, lips, and facial contours (see FIG. 5). For each video frame, the system 131 outputs a complete set of landmark coordinates for the detected face(s). The process is repeated sequentially for all frames, enabling real-time or near-real-time tracking of facial features. Temporal inconsistencies, such as jitter or drift across frames, may occur due to variable lighting, head motion, or occlusions. Therefore, in some aspects, the facial landmark estimates output may be further refined using post-processing techniques such as temporal filtering, statistical smoothing, or machine learning-based correction to improve stability and reliability over time.

Specifically, the ML ingestion layer 129 functions as an intermediate processing stage that enhances landmark stability and anatomical accuracy. The ML ingestion layer 129 is configured to ingest raw landmark data (e.g., typically 468 facial points per frame) output from the facial detection model 127 and passes it through a supervised ML model (MLM) from the real-time facial measurement engine 106. In some aspects, the ML ingestion layer 129 is configured to perform at least one of: filtering out noise, correcting drift, and improving reliability of a set of facial landmark estimates comprising at least pupils, iris edge, a 68-point model, and temple-to-temple width. In some aspects, the ML ingestion layer 129 may generate a reliability score and/or an accuracy score across facial landmark sets. The output of the ML ingestion layer 129 is a refined and validated set of landmarks optimized for measurement precision. In addition, the ML ingestion layer 129 operates as a closed-loop system that continuously refines itself through feedback and ongoing data ingestion, enabling greater measurement accuracy, stability, and repeatability in vision-based facial analysis applications. The resulting data enables frame-accurate tracking of facial geometry, supporting further downstream applications such as measurement of interpupillary distance (IPD), iris diameter, and other pixel-based facial dimensions.

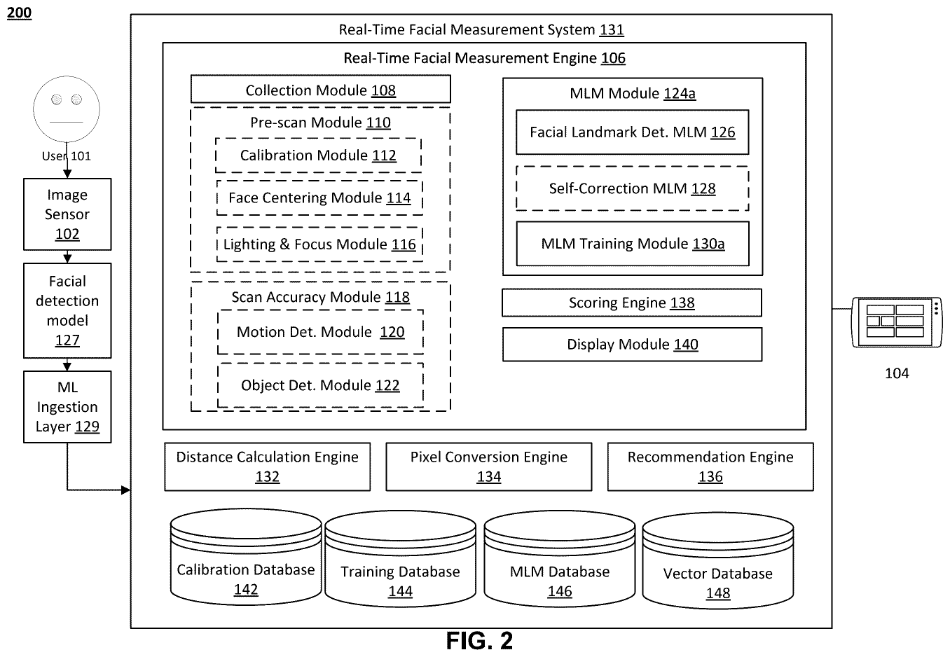

As will be described in more detail in FIG. 2 below, the real-time facial measurement system 131 includes at least a real-time facial measurement engine 106 configured to process video data of the user 101, facial landmark estimates extracted by the facial landmark/detection model 127 and/or refined dataset of the landmark output measurements from the facial detection model 127 to generate facial localization 123, landmark identification 103, and facial alignment 105. The facial localization process involves detecting the presence and position of a face of the user 101 within each frame of the video stream. For example, the facial localization process may involve identifying a bounding region that encapsulates the face using computer vision techniques or deep learning-based object detection models. Following localization, the real-time facial measurement engine 106 performs landmark identification 103 by detecting key facial features, such as the eyes, nose, mouth, and jawline, using a MLM configured to identify anatomical features of a face from frames of video data. These facial landmark estimates enable precise mapping of the user's facial geometry. Subsequently, facial alignment 105 is performed to normalize the orientation of the face. For example, this may include correcting for variations in head tilt, rotation, and scale. This alignment process uses the detected facial landmark estimates to geometrically transform the face to a standard pose, thereby enabling consistent downstream processing such as facial recognition, expression analysis, or biometric measurement.

The outputs generated by the real-time facial measurement engine 106 (e.g., including facial localization 123, landmark identification 103, and facial alignment 105) may be provided as inputs to a distance calculation engine 132 configured to compute precise facial dimensions such as interpupillary distance (IPD) 107, face width 109, nose bridge width 111, and optional temple arm length 137. In some embodiments, the landmark identification 103 process yields a set of spatial coordinates corresponding to predefined anatomical points on the face of the user 101, including the centers of the pupils, outer contours of the cheeks, and boundaries of the nasal bridge. For example, the system 131 may rely on a set of predefined facial landmark indices to accurately identify and track key anatomical regions of the face, which are essential for precise calculations in applications such as eyewear fitting. As will be shown in FIG. 5, critical facial points include the eye corners, iris points, temple to temple width markers, nose bridge points, temple arm length and pupil center points. Each of these indices corresponds to a specific region of the face, providing reliable reference points for determining spatial relationships and dimensions. Because the facial alignment 105 process standardizes the orientation and scale of the facial image data, these landmark coordinates can be reliably interpreted within a normalized reference frame. By leveraging these landmarks, the system 131 ensures accurate and personalized fitting of eyewear based on the user's unique facial structure.

In some aspects, the distance calculation engine 132 utilizes these normalized landmark positions to compute linear distances between specific pairs of points. For example, interpupillary distance 107 is calculated by determining the Euclidean distance between the landmarks corresponding to the centers of the left and right pupils. Similarly, temple to temple width 109 is computed as the distance between the outermost cheek landmarks, and nose bridge width 111 is determined by measuring the distance between the inner eye corners or nasal ridge landmarks. Optionally, a temple arm length 137 may also be calculated from landmarks by estimating a real-world distance along the side of the head from the front of the face near the eye to the back of the ear where the temple arms of glasses rest. These calculations may be performed in pixel space or, in some embodiments, converted to real-world units using a known reference scale or calibration technique by the pixel conversion engine 134. The resulting measurements may be used in downstream applications such as virtual eyewear fitting, biometric analysis, or customized product recommendations.

In some aspects, the interpupillary distance 107, temple to temple width 109, nose bridge width 111, and the optional temple arm length 137 may be provided as inputs to the pixel conversion engine 134 configured to generate landmark-based facial landmark estimates 115 in real-world or metric units (e.g., millimeters). These initial measurements, obtained in pixel space based on the spatial relationships between facial landmark estimates, are typically influenced by factors such as camera resolution, focal length, and user distance from the image sensor 102. The pixel conversion engine 134 may apply a transformation process that converts these pixel-based distances into calibrated metric units (e.g., millimeters or centimeters) by leveraging a reference scaling factor. This scaling factor may be determined through one or more calibration methods, such as using a known reference object within the image frame (e.g., a calibration marker or standardized object), leveraging device-specific camera metadata, or employing depth sensing technologies. Once the conversion factor is established, the pixel conversion engine 134 multiplies the raw pixel distances-such as the pixel-based interpupillary distance-by this factor to yield accurate, real-world measurements. The term “real-world measurements” refers to physical distances or dimensions expressed in standardized units that correspond to actual, physical quantities in the environment, including but not limited to metric units (e.g., millimeters, centimeters, meters, kilometers) and imperial units (e.g., inches, feet, yards, miles). Real-world measurements differ from pixel distances, which represent discrete counts of pixels within a digital image or display and lack intrinsic physical scale absent a defined conversion factor. The resulting landmark-based facial landmark estimates may include, but are not limited to, calibrated interpupillary distance 107, anatomical temple to temple width 109, nose bridge width 111, or optional temple arm length 137. These measurements can then be used in applications requiring precise sizing or fitting, such as augmented reality (AR) overlays, virtual try-on systems, biometric identification, or personalized wearable product recommendations.

In some embodiments, landmark-based facial estimates 115 and optional prescription scan and ingestion data are provided as inputs to a recommendation engine 136 configured to generate prescription-related measurement 117, perform face and frame matching 119, and output a personalized eyewear recommendation 121. The landmark-based facial estimates 115 may include, but are not limited to, IPD, temple to temple width, nose bridge width, ocular center height, and segment height, each derived from real-time facial landmark analysis. In some aspects, the optional prescription scan and ingestion module 113 may obtain prescription data from user-uploaded documents, electronic medical records, or images processed using optical character recognition (OCR). The optional prescription scan and ingestion module 113 is also configured to store both dynamic facial measurements and static frame measurements captured from selected video frames or high-resolution still images. These data inputs are used to generate advanced optical fitting parameters, including segment height (representing the vertical distance from the bottom edge of the virtual frame during scanning to the start of the near-vision zone in multifocal lenses) and ocular center height (indicating the vertical positioning of the pupil within the lens area).

The recommendation engine 136 uses this information to generate a prescription-related measurement 117 that ensures proper optical alignment based on both the user's prescription and their unique facial structure. Subsequently, the recommendation engine 136 conducts face and frame matching 119 by analyzing the compatibility of the facial measurements of the user 101 with a database of eyewear frames, evaluating parameters such as bridge width, lens height, frame width, and the vertical alignment of lenses with ocular centers. Based on these calculations and compatibility assessments, the recommendation engine 136 generates a personalized eyewear recommendation 121 optimized for the user's prescription accuracy, facial anatomy, comfort, and aesthetic fit. This recommendation may include specific frame models, lens configurations, and fitting adjustments, enabling a comprehensive and customized eyewear solution.

The applications of the real-time facial measurement system 131 may include at least ensuring bifocal and progressive lens are accurately positioned for vision correction, ensures eye tracking aligns properly with virtual reality headsets, ensures correct face symmetry analysis for reconstructive surgery, and tracks eye position in real-time to detect driver fatigue and distractions. By factoring in individual characteristics, the system 131 ensures a highly accurate and personalized eyewear recommendation. In some aspects, the system presents an eyewear selection ranked by percentage fit accuracy. Each frame is displayed hierarchically based on its form, fit, and function relative to the user's unique facial structure. The system may continuously improve through: (1) self-learning AI algorithms that refine recommendations over time, (2) real-time data ingestion and processing, and (3) enhanced predictive modeling for future frame styles and fits. By leveraging generative AI, the system 131 evolves dynamically with every new scan to constantly improve its precision and adaptability.

The described system and methods provide several technical benefits that enhance the precision, efficiency, and personalization of eyewear fitting and prescription recommendation processes. For example, by leveraging real-time facial landmark detection, facial alignment, and pixel-to-metric conversion, the system enables accurate extraction of anatomical measurements such as interpupillary distance 107, temple to temple width 109, nose bridge width 111, optional temple arm length 137, segment height, and ocular center height, without requiring physical measurement tools or in-person fittings. The integration of prescription scan and ingestion functionality further streamlines the user experience by automating the capture and structuring of prescription data from various input sources, including scanned documents and electronic records. The combination of these data points within the recommendation engine 136 facilitates highly accurate prescription-related calculations, precise alignment of optical centers with user-specific eye positions, and optimal selection of eyewear frames based on individualized facial geometry. This results in improved visual clarity, comfort, and aesthetic appeal for the user 101. Additionally, the system reduces reliance on manual fitting methods, minimizes the potential for human error, and enables scalable, remote delivery of customized eyewear solutions, thereby improving accessibility and efficiency across tele-optometry and e-commerce eyewear platforms.

It should be noted that the various processes described in system 100 are described at a high level and further detail about the specific steps and processes will be described in further detail below.

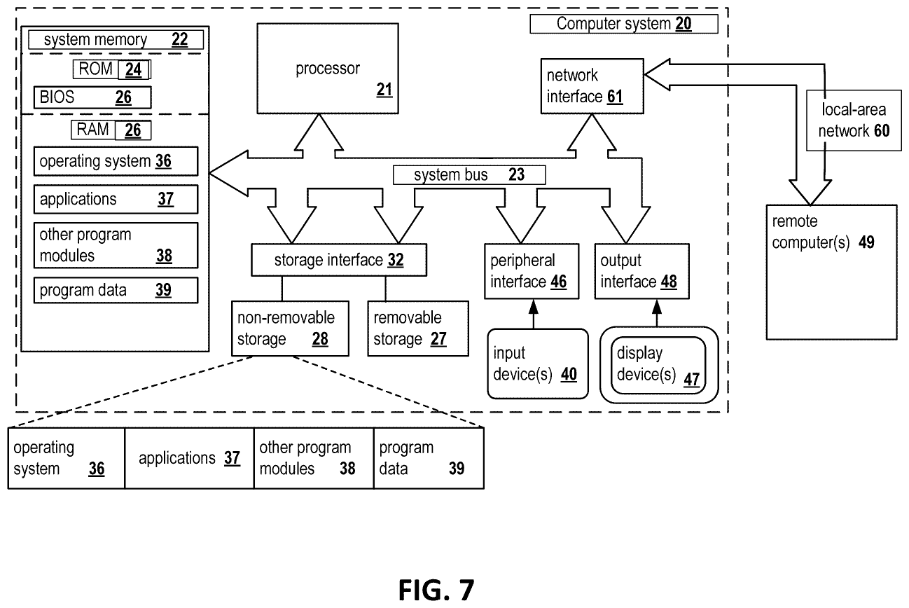

FIG. 2 is a block diagram illustrating a system for determining prescription-related facial measurements for fitting eyewear according to aspects of the present disclosure. In one aspect, the components of system 200 may be implemented on computer systems, such as that shown in FIG. 7.

The present disclosure describes a real-time facial measurement system 131 comprising at least a real-time facial measurement engine 106 for detecting facial measurements using a deep learning-based MLM. Pre-trained on millions of facial images, the facial detection/landmark models 127 may detect 478+ facial landmark estimates in real time, covering key facial features such as the eyes, nose, mouth, and chin to enable comprehensive structural analysis. Unlike conventional systems that only capture 30 frames per second (FPS), the system 200 processes 60-240 FPS to continuously track micro-movements and reduce errors from motion blur or occlusions. Key frames are then selected using a weighted algorithm, and the coordinates of critical landmarks (e.g., eye corners, iris points, temple to temple width, nose bridge, and pupil center) are extracted for precise measurement. For example, using a smartphone camera (e.g., computing device 104 and image sensor(s) 102) operating at 120 FPS, the system 200 captures facial data, detects landmarks, and stores the (x, y) coordinates from the most accurate frames. To ensure sub-millimeter accuracy, the system 200 may optionally incorporate a calibration phase and real-time validation to verify optimal user position, lighting, focus, and stability. The scan pauses if conditions fall outside defined thresholds, preventing errors caused by poor positioning, excessive movement, blurriness, or foreign objects in the frame.

The system 200 may then be used to calculate prescription-specific facial measurements to facilitate precise and personalized eyewear fitting based on user-specific anatomical data. The real-time facial measurement engine 106 is configured to capture video data of a face of the user 101 from an image sensor 102 via the ML ingestion layer 129 and/or the facial detection model 126 extract facial landmark estimates using a pre-trained facial landmark detection MLM 126 or the facial detection model 127, calculate Euclidean distances between selected landmark pairs, and generate prescription-related measurements including, but not limited to, temple to temple width, eye distance, temple arm length, and nose bridge width for personalized eyewear recommendation. Thus, the real-time facial measurement engine 106 may detect key facial landmark estimates with high precision to generate measurements of a face of the user 101, which is essential for both prescription-related measurements and eyewear fitting.

The system 200 includes at least a computing device 104, at least one image sensor 102 (e.g., as part of the computing device 104 or coupled to the computing device 104), a facial detection model 127, a ML ingestion layer 129, a real-time facial measurement system 131 including a real-time facial measurement engine 106, a distance calculation engine 132, a pixel conversion engine 134, a recommendation engine 136, and a computing device 104. The facial detection model 127 may be configured to identify key facial regions and landmarks with high precision and low computational overhead by utilizing pre-trained machine learning model. In some aspects, the facial detection model 127 employs a pipeline architecture that includes face region proposal, landmark regression, and temporal smoothing to enable continuous and stable face tracking across frames. The ML ingestion layer 129 is configured to filter out noise, correct drift, and/or improve the reliability of the key facial regions and landmarks from the facial detection model 127. The real-time facial measurement engine 106 may be configured to generate prescription-related measurements for recommending eyewear based on video data capturing the face of the user 101. The computing device 104 may execute a plurality of modules in the real-time facial measurement engine 106 that together perform collection, calibration, analysis, self-correction, detection, calculation, conversion, and recommendation functions. In some aspects, the real-time facial measurement engine 106 may correspond to the computing device 104 or cloud network (not shown) that is configured to execute a plurality of modules that together make up the real-time facial measurement engine 106 for generating prescription-related measurements.

At a high level, the image sensor 102 captures video data of the user's face for continuous facial landmark tracking. Specifically, the facial detection model 127 processes each video frame captured from the image sensor 102 to identify a region of interest (e.g., a face of the user) and extract hundreds facial landmark estimates in real-time per frame. Each landmark includes normalized x and y coordinates (relative to the frame) and a z coordinate indicating relative depth. These landmarks correspond to key anatomical features such as the eyes, nose, lips, and facial contours (see FIG. 5). For each frame, the facial detection model 127 outputs a complete set of landmark coordinates for all detected faces. This process repeats across frames to enable real-time or near-real-time facial tracking.

To implement a system and method for training and performing inference with the facial detection model 127 for face detection, a computing apparatus is provisioned with instructions that enable execution of a pipeline-based architecture comprising modular and interdependent components, referred to as “calculators.” The process begins with sourcing a labeled dataset comprising a plurality of human face images, each annotated with bounding box coordinates representing facial regions. Optionally, these images may be preprocessed to ensure uniform dimensions and normalized pixel values. For the training phase, a neural network architecture, such as a convolutional neural network (CNN), is initialized with random weights or pretrained on a generic vision task. The annotated dataset is input into the training pipeline, wherein the network parameters are iteratively updated via gradient descent optimization to minimize a loss function defined by the difference between predicted and ground truth face locations.

Upon completion of the training phase, the optimized model is exported into TensorFlow Lite or a compatible format that adheres to inference requirements of the facial detection model 127. In the inference phase, the model is embedded within a facial detection model 127 graph comprising a sequence of calculators, including but not limited to image preprocessing calculators, inference calculators, and postprocessing calculators. During runtime, an input video or image stream is passed to the graph, wherein each frame is first normalized and resized, then processed by the inference calculator employing the trained face detection model. The output of the inference calculator is subjected to non-maximum suppression and thresholding by postprocessing calculators to eliminate redundant detections and isolate salient face bounding boxes. In some aspects, the facial detection model 127 outputs 468 facial landmark estimates, but some of those shift or jitter across frames. To address this, the ML ingestion layer 129 pulls in those landmarks frame by frame and passes them through a validation and filtering pipeline. This system 200 tracks landmark stability over time using techniques such as rolling averages and Kalman filters, enabling it to discard outliers that do not conform to predicted anatomical movement patterns. The final detection and filtered landmark results are subsequently rendered or utilized for downstream applications such as facial landmark refinement, pose estimation, or emotion recognition. The system 200 operates in real-time on both mobile and desktop platforms by leveraging hardware acceleration and efficient graph execution paradigms inherent to the facial detection model 127 framework.

The ML ingestion layer 129 functions as an intermediate processing stage that improves the stability and anatomical accuracy of facial landmark estimates from the facial detection model 127 for more accurate and reliable downstream processing. Specifically, the ML ingestion layer 129 receives raw landmark data and processes them by applying temporal analysis techniques to track facial landmark behavior across frames, reduce noise, correct drift, and/or remove outliers. In some aspects, the ML ingestion layer 129 may also score landmark sets for reliability and accuracy. The output is a refined, validated set of facial landmark estimates optimized for high-precision measurement. Additionally, the ML ingestion layer 129 functions as a closed-loop system, continuously improving through feedback and ongoing data ingestion. This enables frame-accurate facial geometry tracking and supports downstream applications such as precise measurement of IPD, iris diameter, and other pixel-based facial dimensions.

In some aspects, the real-time facial measurement engine 106 may include a collection module 108, an optional pre-scan module 110 including at least an optional calibration module 112, an optional face centering module 114, and an optional lighting and focus module 116, an optional scan accuracy module 118 including an optional motion detection module 120 and an optional object detection module 122, an optional self-correction MLM 128, a MLM module 124a including a facial landmark detection MLM 126, an optional self-correction MLM 128, and a MLM training module 130a, a scoring engine 138, and a display module 140, a distance calculation engine 132, a pixel conversion engine 134, a recommendation engine 136, and a computing device 104. In some aspects, pre-scan measurements (e.g., user distance, face centering, lighting, and/or focus measurements) that ensure optimal scanning conditions may be stored on a calibration database 142. In some aspects, training data for the MLM training module 130a may be stored on a training database 144. In some aspects, a pre-trained facial landmark detection MLM 126 may be stored on a MLM database 146. In some aspects, the calibration database 142, training database 144, the MLM database 146, and/or the vector database 148 may be stored on a local device or a cloud network.

The computing device 104 may execute a collection module 108 to obtain a set of video data via one or more image sensors 102 (e.g., from a smartphone or webcam) to continuously capture the face of a user 101 for high-precision facial landmark tracking in conjunction with the facial detection model 127. By utilizing advanced facial landmark processing via the facial detection model 127, the system 200 performs high-frequency frame analysis at 60 to 240 frames per second (FPS), significantly exceeding the 30 FPS limit of older technologies and enabling finer detection of facial details. This elevated frame rate enables real-time, frame-by-frame landmark validation and correction, allowing the system 200 to accurately track even the smallest micro-movements of the face of the user 101. In this way, the system 200 may analyze multiple frames in sequence and selectively extract key frames with the clearest facial positioning to reduce the impact of motion blur and/or partial occlusions. In some aspects, the collection module 08 may extract facial landmark data measured in a pixel-space from the refined dataset of landmark output measurements, wherein the facial landmark data comprises at least one of: iris boundary, centers of pupils, midpoints between an upper and lower eyelid landmark, a 68-point model, or a widest part of the face. This continuous, high-speed video capture ensures precise and reliable facial measurements, enhancing the overall effectiveness of the system 200.

The computing device 104 may execute an optional pre-scan module 110 to optimize scan conditions before the user begins recording their face for obtaining facial measurements. Specifically, the optional pre-scan module 110 may initiate a series of pre-scan calibration procedures designed to optimize environmental and user-related conditions prior to facilitate sub-millimeter precision in facial measurements. These preparatory measures ensure that the user 101 is correctly positioned at an appropriate distance and angle relative to the imaging sensor 102, while also verifying sufficient lighting and camera focus. The system 200 systematically evaluates multiple variables, including user proximity, facial alignment, ambient illumination levels, and image sharpness, to confirm that scanning conditions meet established quality benchmarks. This pre-scan validation process mitigates common sources of error such as improper user distance, blurriness, inadequate lighting, excessive head tilt, or visual obstructions within the frame. By enforcing these quality gates before initiating the scan, the system 200 significantly reduces the likelihood of distortion and ensures that only high-fidelity image data is captured for subsequent facial analysis and measurements.

Prior to initiating a facial scanning procedure, the system 200 may enter a calibration phase to verify that the user's position, distance, and facial orientation relative to the image-capturing device fall within predetermined optimal parameters. Specifically, an optional calibration module 112 may identify facial landmark estimates and determines a bounding box around the face of the user 101 to assess alignment within a defined spatial zone conducive to accurate biometric measurement. In addition, the optional calibration module 112 may also compute the real-world distance of the user 101 from the one or more image sensors 102 by estimating the interpupillary distance (IPD) in pixels and comparing it to an expected average IPD value (e.g., 64 mm). For example, the estimated distance may be computed as the known IPD x camera focal length divided by the detected IPD in pixels. In response to detecting a deviation from a predefined distance threshold (e.g., when the estimated user-to-camera distance is less than approximately 30 centimeters or exceeds approximately 80 centimeters), the system may activate a control routine configured to suspend the scanning operation. Concurrently, the system 200 may generate one or more user-facing prompts, which may be visual and/or auditory in nature, instructing the user 101 to adjust their position to fall within the acceptable distance range. Such calibration ensures that facial scans are conducted under conditions that minimize distortion and maximize reliability of biometric output.

The optional face centering module 114 may also implement a real-time facial bounding box tracking mechanism to continuously monitor the position of the user's 101 face relative to the center of the imaging frame. In some aspects, an artificial intelligence (AI) module such as the facial landmark detection MLM 126 may actively analyzes facial landmark data to ensure the face remains centrally aligned within a predefined bounding region. As an example, if more than approximately 30% of the facial area deviates beyond the bounds of this region, the system may initiate an automated interruption protocol that temporarily pauses the scanning process and presents the user with a prompt-visual, auditory, or both-instructing them to re-center their face. In parallel, a mathematical out-of-frame detection algorithm may compute the horizontal offset of the face center from the center of the display. As another example, if the calculated offset exceeds a threshold of approximately 30% of the total screen width, the system 100 flags an out-of-frame error and suspends the scan. Yet another example, on a display with a width of 1080 pixels, a detected face center at 900 pixels yields an offset of (900-540)/1080=33%, exceeding the threshold, and resulting in a scan pause and user guidance to reposition accordingly.

The optional lighting and focus module 116 may perform a pre-scan evaluation of lighting conditions and image focus clarity to optimize image fidelity and ensure the highest level of scan accuracy. As part of this process, the system 200 assesses the average luminance (L) within the detected facial region. If the measured brightness falls below a lower threshold (e.g., L<50, indicating insufficient lighting) or exceeds an upper threshold (e.g., L>220, indicating overexposure), the system 200 halts the scanning sequence and issues a prompt instructing the user 101 to adjust the ambient lighting conditions. For instance, a measured brightness level of 30 would trigger the system 200 to pause and display a “Move to Better Lighting” notification. In addition to lighting assessment, the system 200 applies a blur detection algorithm based on Laplacian Variance analysis to evaluate image sharpness. If the resulting sharpness metric falls below a predetermined threshold (e.g., variance <1000), indicative of a blurry image, the system interrupts the scan and generates a prompt advising the user to refocus the camera or stabilize their position. As an example, a calculated variance of 800 would activate this safeguard, ensuring that only well-illuminated and sharply focused images are processed for biometric analysis.

The computing device 104 may execute an optional scan accuracy module 118 to employ, during the facial scanning process, a real-time accuracy enforcement mechanism to continuously evaluate frame quality and ensure adherence to precision standards required for sub-millimeter facial measurement accuracy. The system 200 may actively monitor a range of dynamic conditions, including user position, facial clarity, stability, and the presence of foreign objects within the capture frame. If any frame fails to meet predefined quality thresholds (e.g., such as improper user distance, motion-induced blur, lighting inconsistencies, off-angle facial orientation, or obstructions affecting landmark detection), the system 200 temporarily halts the scan and prompts corrective user action before resuming. By filtering out suboptimal frames in real time, the system 200 ensures that only high-fidelity data is processed, thereby minimizing distortion and maximizing the integrity of biometric output.

Specifically, the optional motion detection module 120 may continuously monitor facial stability. The optional motion detection module 120 may utilize the facial landmark detection MLM 126 to track key facial landmark estimates across sequential image frames to calculate displacement vectors to assess the degree and speed of user movement. For example, if the displacement of tracked points exceeds a predefined threshold (e.g., 2 millimeters between consecutive frame), the system 200 automatically pauses the scan to prevent data distortion. Additionally, the system 200 evaluates the velocity of motion (e.g., Motion Speed=(d_{frame2}−d_{frame1})/Δt), and if the calculated movement speed exceeds approximately 3 millimeters per second, it flags the activity as excessive and prompts the user to remain still. For example, if the user shifts their head laterally by 4 millimeters within a time span of 0.1 seconds, the scan is interrupted, and the user is instructed to stay still. This enforcement mechanism ensures that only stable, high-quality frames are captured for biometric processing.

In addition, the optional object detection module 122 may identify and manage the presence of foreign objects (e.g., hands, glasses, or mobile phones) in the video frames to ensure accurate facial landmark detection. In some aspects, this process begins with object detection using the YOLOv5 model, which classifies all visible objects in the camera view. If non-facial objects are detected, the system 200 automatically pauses the scan to prevent interference. In addition, the system 200 includes occlusion handling. For example, if more than 20% of the face is obscured, the scan is halted, and the user is prompted to remove the obstruction. As another example, if glasses are detected during a scan not requiring them (e.g., a non-prescription scan), the system 200 will pause and notify the user to take them off before proceeding.

The computing device 104 may execute the optional self-correction MLM 128 to compensates for common user-induced variations such as head tilts, minor movements, blinking, or partial eye closures, as well as environmental factors like lighting fluctuations. Specifically, the optional self-correction MLM 128 may operate through a continuous, real-time feedback loop that ensures high precision even in dynamic or imperfect conditions. As the user's face is scanned, the system 200 tracks key facial landmark estimates and monitors for disruptions caused by factors such as head tilt, slight movements, blinking, partial eye closure, or changes in lighting. When the optional self-correction MLM 128 detects landmark drift (e.g., significant deviations from expected positions, it automatically triggers a secondary validation scan. This secondary scan may use a refined model or select a clearer frame to reassess and recalibrate the landmark positions. The corrected data is then reintegrated into the active scan, allowing the system 200 to maintain accurate facial mapping without requiring user input. This adaptive, self-correcting mechanism ensures consistent and reliable results, even in less-than-ideal scanning environments.

The computing device 104 may execute the MLM module 126 including at least a facial landmark detection MLM 126, an optional self-correction MLM 128, and a MLM training module 130a. The facial landmark detection MLM 126 is configured to identify anatomical features of a face for the user from each frame of the set of video data. In addition, the facial landmark detection MLM 126 is also configured to select pairs of facial landmark estimates (e.g., center of left pupil and center of right pupil, a first outer iris edge and a second outer iris edge, or a first nose bridge point and a second nose bridge point).

There are several possible approaches that may be implemented using computer vision and MLMs such as a neural network (e.g., a convolutional neural network (CNN) and/or a Graph Neural Network (GNN)) with a real-time, self-adjusting AI model. This hybrid approach enables the facial landmark detection MLM 126 to not only extract and interpret complex facial features with high accuracy, but also adapt dynamically to changes in user behavior or environmental conditions, ensuring continuous precision throughout the scanning process.

A neural network is a type of machine learning process that uses interconnected nodes or neurons in a layered structure that resembles the human brain. The neural networks create an adaptive system that computers use to learn from their mistakes and improve continuously by comprehending unstructured data and make observations without explicit training. With neural networks, computers may distinguish and recognize images similar to humans. However, the neural networks in the facial landmark detection MLM 126 must first go through training to teach the neural networks to perform their respective specific tasks. It should be noted that CNN or GNN is described in this present disclosure for illustrative purposes only and that any suitable MLM may be utilized to perform the specific tasks of the facial landmark detection MLM 126.

The MLM module 124a may comprise one or more neural networks, which are a class of MLMs inspired by the structure and functioning of the human brain. They consist of interconnected nodes, called neurons or artificial neurons, organized into layers. Neural networks are capable of learning complex patterns and representations from data. The neural network executed by MLM module 124a may be one of the following: transformer neural network, convolution neural network (CNN), Graph Neural Network (GNN), recurrent neural network (RNN), long short-term memory (LSTM) network, gated recurrent unit (GRU) network, autoencoder, generative adversarial network (GAN). CNNs are effective for image-related tasks because CNNs may automatically learn spatial hierarchies of features from the input images. For videos, RNNs or Long Short-Term Memory (LSTM) networks can be used to capture temporal dependencies between frames. In some aspects, a hybrid model may be used by combining CNNs for spatial feature extraction and RNNs for temporal analysis.

A CNN is specialized for processing grid-like data, such as images, and employs convolutional layers to learn spatial hierarchies of features, reducing the need for manual feature engineering. CNNs are well-suited for tasks like image classification, object detection, and image generation.

A GNN is a deep learning model that understands and learns from the connections between things, not just the things themselves. It's ideal when the relationships between data points are just as important as the data points

An autoencoder is a type of neural network used for unsupervised learning and dimensionality reduction and consists of an encoder that compresses input data into a lower-dimensional representation (encoding) and a decoder that reconstructs the original input from the encoding.

A GAN comprises a generator and a discriminator trained simultaneously through adversarial training. The generator aims to generate realistic data, while the discriminator tries to distinguish between real and generated data. A GAN is widely used for image and content generation tasks.

For computer vision tasks such as extracting a set of facial landmark estimates within video frames, an untrained facial landmark detection MLM 126 will first analyze the images from the training dataset (e.g., training database 144) to learn how to identify facial landmark estimates on the face of the user 101. As an example, the training dataset may include labeled dataset containing images or videos of faces of different people under different lighting conditions and varying color temperature.

During training of the facial landmark detection MLM 126, the training dataset will include at least facial images annotated with a distance between an outer or inner corners of both eyes of a user; facial images annotated with left and right boundaries of each iris; facial images annotated with a pupil center for each eye; facial images annotated with landmarks at a widest visible part of the face; and facial images annotated with a nasion point or a bottom of a nose bridge. The results from the untrained facial landmark detection MLM 126 is then compared with known data set results using corresponding labels identifying at least a distance between an outer or inner corners of both eyes, left and right boundaries of each iris, a pupil center for each eye, a widest visible face of the user, or a nasion point or a bottom of a nose bridge. It should be noted that the input to the facial landmark detection MLM 126 will be the images from the training dataset 144.

For every input training sample from the training dataset, the neural network from the facial landmark detection MLM 126 will produce a prediction consisting of values representing the probability that a facial landmark detected is detected on an image of the face. The output with the highest probability determines the predicted pass or fail label. A class label for each input image is used to compute a loss (e.g., loss function).

The facial landmark detection MLM 126 then uses a loss function that quantifies the error between the predicted output and the ground truth for a given training sample. In other words, the loss function can be used to guide the learning process by updating the network weights in a way that improves the accuracy of future predictions. This process may continue until the difference between the prediction and the correct targets is minimal. In some examples, an appropriate loss function, such as Mean Squared Error (MSE) for regression tasks (e.g., predicting brightness levels) or a Cross-Entropy Loss for classification tasks (e.g., detecting specific color changes).

In some aspects, an optimizer such as Adam or SGD may be used to train the models in the facial landmark detection MLM 126. In some aspects, the data may be split into training, validation, and test sets. In these aspects, the models from detection are trained on the training dataset and then validated by the validation sets to tune hyperparameters.

Once the neural network is trained (e.g., inference), the facial landmark detection MLM 126 may extract a set of facial landmark estimates from a frame containing the face of the user 101. Specifically, the facial landmark detection MLM 126 may contain a trained neural network configured to identify and detect facial landmark estimates in video frames capturing the face of the user.

During inference, the trained neural network model from facial landmark detection MLM 126 does not re-evaluate or adjust the layers of the neural network based on the results.

Instead, the inference applies knowledge from the trained neural network and uses it to infer a result. Accordingly, when a new unknown dataset (e.g., video stream) is input through the trained neural network in the facial landmark detection MLM 126, the trained neural network outputs a prediction of the presence of a facial landmark based on its trained predictive accuracy of the neural network.

The computing device 104 may execute a distance calculation engine 132 to compute the Euclidean distance between each selected pair of facial landmark estimates by first receiving the coordinates (typically x, y) of the landmarks identified on a facial image. For each specified pair, the distance calculation engine 132 applies the Euclidean distance formula, which involves calculating the square root of the sum of the squared differences between the corresponding coordinates of the two points. Specifically, for a pair of landmarks with coordinates (x₁, y₁) and (x₂, y₂), the distance calculation engine 132 computes the distance as √[(x₂−x₁)²+(y₂−y₁)²]. As an example, if the detected pupil center has the following coordinates: left pupil x₁, y₁)=(350, 220) and the right pupil (x₂, y₂)=(750, 220). The system may calculate the distance as 400 pixels. This process is repeated across all selected pairs, enabling precise measurement of spatial relationships between key facial features for use in downstream processing tasks such as expression analysis, identity verification, or facial symmetry evaluation.

In some aspects, the computing may also execute a distance calculation engine 132 to generate a reliability score and/or an accuracy score for each facial landmark derived from the refined dataset of landmark output measurements. Specifically, the distance calculation engine 132 is configured to process a refined dataset of facial landmark output measurements to generate one or more quality metrics for each identified facial landmark. For example, the distance calculation engine 132 may compute spatial or feature-based distances between the detected landmarks and reference positions or geometric relationships expected among a typical set of facial features. Based on these calculations, the engine may determine a reliability score indicative of the confidence or trustworthiness of each landmark, as well as an accuracy score representing the estimated deviation from a predicted or ground truth location. These scores may be utilized to evaluate the overall integrity of the landmark data, filter or weight landmark inputs during downstream processing, or trigger corrective actions in subsequent modules of the facial analysis system. By quantifying the precision and dependability of each landmark, the system 200 may enhance the robustness and reliability of facial recognition, expression analysis, or tracking applications.

The computing device 104 may execute a pixel conversion engine 134 to convert pixel distances of facial measurements for the user into real-world millimeter distances by applying a predefined scaling factor to generate a real-world facial fit measurement comprising at least one of an iris width, IPD distance, a nose bridge width distance, or temple to temple distance. This scaling factor represents the ratio of millimeters to pixels and is typically derived from a known reference measurement on the user's face, such as the distance between the pupils or another anatomically consistent feature. Once the scaling factor is established, the pixel conversion engine 134 multiplies each pixel distance (previously calculated between selected facial landmark estimates) by this factor to obtain the corresponding measurement in millimeters. This conversion enables accurate, real-world interpretation of facial dimensions, which can be critical for applications such as custom eyewear fitting, medical diagnostics, or biometric analysis.

The computing device 104 may execute a recommendation engine 136 to generate prescription-related measurements for recommending eyewear by utilizing the calculated Euclidean distances between key facial landmark estimates in combination with results from a pre-trained MLM. These Euclidean distances, derived from the user's facial features, are first processed to extract critical metrics such as temple to temple width, eye distance (IPD), temple arm length, and nose bridge width. The pre-trained MLM is then used to interpret these measurements within the context of known anatomical patterns and eyewear fitting data, enabling the engine to produce personalized prescription-related recommendations. By integrating geometric analysis with learned patterns from a dataset of facial structures and corresponding eyewear prescriptions, the engine ensures that the generated measurements are both anatomically accurate and optimized for comfort and fit.

The computing device 104 may execute a display module 140 to generate and display the prescription-related measurements on the computing device 104. Generally, the display module 140 is responsible for managing and rendering the visual components of the user interface by handling the presentation of information to the user, ensuring that data and controls are displayed correctly and consistently across the UI.

In some aspects, the display module 140 is configured to render or draw all the elements of the UI, such as windows, buttons, text fields, menus, icons, images, and other components. In some aspects, the display module 140 is configured out update the UI when the data changes or user interactions occur (e.g., clicking a button or typing in a text box) such that the display module updates the UI accordingly. This could mean refreshing a portion of the screen, changing the state of a button, or displaying new data. In other words, the display module 140 may be considered the “view” part of a model-view-controller (MVC) or similar design pattern. It serves as the layer that presents data to the user and receives input to and from the computing device 104.

In this way, the real-time facial measurement engine 106 ensures the reliability and integrity of real-time facial measurement. For example, the real-time facial measurement engine 106 may integrate one or more scanning checks (e.g., distance detection, face centering validation, lighting optimization, blur detection, motion stability enforcement, and foreign object filtering) to enhance the accuracy of image capture and analysis. Each factor may address a challenge in real-time environments: distance detection ensures the subject is positioned appropriately; face centering validation guarantees the face is fully within frame; lighting optimization adjusts for ideal illumination; blur detection filters out unclear images; motion stability enforcement minimizes the effects of movement; and foreign object filtering prevents obstructions from skewing results. Together, these checks reduce errors, improve consistency, and reinforce security by ensuring that only high-quality, valid data is used in verification or recognition tasks.

In addition, the real-time facial measurement engine 106 also enhances the integrity and quality of real-time scanning processes. Unlike traditional systems that continue capturing data even when conditions are suboptimal (leading to inaccurate results or failed verifications) this system 200 proactively monitors scan quality in real time. When it detects that conditions such as lighting, focus, or alignment fall below acceptable thresholds, it automatically pauses the scanning process to prevent the collection of poor-quality data. More importantly, it provides users with real-time guidance on how to correct the issue, such as adjusting their position or improving lighting. This not only enhances user experience by reducing frustration and repeated attempts but also ensures that only high-quality, reliable scans are used for further processing, ultimately increasing accuracy and system trustworthiness.

It should be noted that the collection, calibration, detection, self-correction, analysis, calculation, and recommendation modules described in the present disclosure are heavily simplified. One skilled in the art will appreciate that the MLMs utilized may have significantly large datasets with highly specific details far beyond the capabilities of the human mind and cannot be realistically performed using pen and paper due to the complexity, precision, and real-time processing requirements involved. These modules may leverage high-speed AI models to detect over 478 facial landmark estimates with sub-millimeter accuracy across hundreds of frames per second-something the human eye and hand cannot track or replicate. Calibration involves dynamically measuring distance and angle between the face and the camera using pixel-based estimations of interpupillary distance, while detection systems simultaneously account for user motion, lighting conditions, focus sharpness, and facial occlusions. The optional self-correction MLM 128 automatically adjusts for landmark drift using multi-frame consistency checks and revalidation processes, which would be infeasible to manage manually. Further, calculations use precise Euclidean distance formulas and conversion algorithms that depend on dynamic scaling factors. Finally, the recommendation engine 136 integrates these measurements with outputs from a pre-trained MLM trained on vast, diverse datasets-tasks requiring data processing and pattern recognition at a scale and complexity unattainable by human effort or traditional tools.

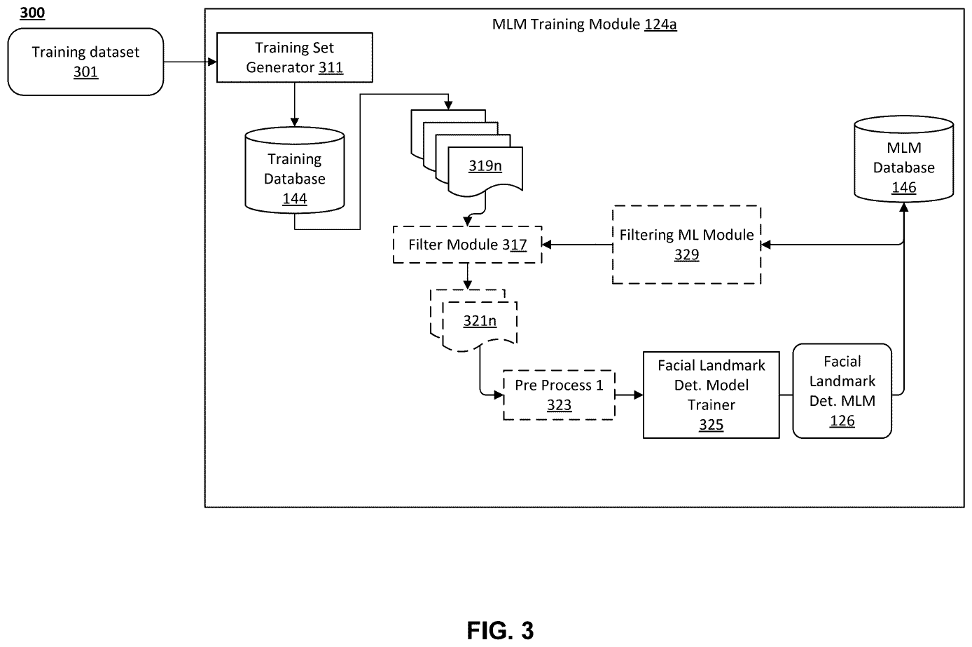

FIG. 3 is a block diagram illustrating a system for preparing an MLM to extract a set of facial landmark estimates and to select pairs of facial landmark estimates according to aspects of the present disclosure.

As shown in example 300, the MLM training module 124a is configured to build and train specialized MLMs with inference to perform particular tasks. This enables the specialized MLMs to develop an ability to perform particular objectives on inputs that are not part of a training dataset. By subjecting the specialized MLMs to large amounts of unlabeled and/or labeled training data sets, the specialized MLMs may perform particular tasks such as evaluating a presentation by generating a presentation score, evaluating an engagement of the audience members by generating an engagement score, and/or identifying a subject matter for the presentation.

Supervised learning is effective for tasks such as classification (assigning inputs to predefined categories) and regression (predicting continuous values) since it relies on the availability of labeled data for both training and evaluation phases. In supervised learning, the MLM training module 124a trains the algorithm on a labeled dataset, where each input has a corresponding output. The goal is to learn a mapping function from inputs to outputs, allowing the algorithm to make predictions or classifications on new, unseen data. The process typically involves the following steps: training, model building, prediction, feedback, and adjustment. In the training phase, the MLM training module 124a provides the algorithm with a training dataset including input-output pairs. The algorithm learns the mapping function that relates inputs to outputs through an iterative process, adjusting its internal parameters based on the provided examples.

During model building, the algorithm creates a model that can generalize from the training data to make predictions on new, unseen data. The model's complexity varies based on the algorithm used. For example, the model may be a simple linear regression model or a complex neural network. During the prediction phase, the MLM training module 124a inputs test inputs (i.e., inputs with known outputs) into the model, which generates predictions or classifications based on what it has learned during training. The accuracy of predictions is evaluated by comparing them to the known outputs in a validation or test dataset. During the feedback and adjustment phase, machine refines the model based on feedback from its predictions. If the predictions differ from the actual outputs, the algorithm adjusts its internal parameters to minimize the errors. The performance of the trained model is assessed using metrics such as accuracy, precision, recall, etc., depending on the nature of the problem.

In some aspects, the MLM training module 124a includes at least a training database 144 configured to store the raw training data 319n and corresponding labels, a MLM database 146 to store the trained models (e.g., the trained facial landmark detection MLM 126). In some aspects, the MLM training module 124a may include an optional filtering ML module 329 and an optional filter module 317 configured to filter data from the training database 144 for training by removing poorly generated training data.

Training data from the training dataset 301 is received into the MLM training module 124a via the training set generator 311. Details about the data included in each training dataset is described in more detail above with FIG. 1.

An optional filtering ML module 329 is configured to filter out bad training images and/or data in order to clean up the training data in the training dataset 319n. In some examples, the optional filter module 317 may be a neural network. In some examples, the optional filter module 317 is a mathematical model. In some examples, the cleaned training dataset 321n then undergoes optional preprocessing steps depending on which neural network or model is being trained.

The optional preprocess 1 223 is an automated process that modifies the raw data received from 319n (or cleaned training dataset 321n) and prepare the raw data as input to the facial landmark detection model trainer 325. These may be described in the MLM training module 124a as snippets of code that prepares the datasets. In some examples, the preprocessing module (e.g., preprocess 1 223a) for a particular trainer may be an automated script or code that will be setup the first time any model is trained.

The facial landmark detection model trainer 325 is a script or code that train the facial landmark detection MLM 126. The facial landmark detection model trainer 325 may be a script or code that holds the instructions on how a model should be trained (e.g., optimization method, model architecture, dataset division, etc.) and runs the training. The facial landmark detection model trainer 325 take as input the raw or filtered processed training data and train the facial landmark detection MLM 126 to achieve their specific objectives, respectively.

In summary, the raw dataset 319n or cleaned dataset 321n may optionally go through a preprocessing step 323 and then a corresponding facial landmark detection model trainer 325 to generate a pre-trained facial landmark detection MLM 126. In some examples, each of these models may be an MLM or a neural network.

As a non-limiting example and as discussed above, the machine learning may be a neural network. The neural network models are designed using a set of hyperparameters that define high-level aspects of their architecture and training process. These hyperparameters include but are not limited to a combination of architecture type, number of layers, memory size, number of attention heads, learning rate, batch size, optimization algorithm, and the like. Based on these hyperparameters, learnable variables called parameters are initialized, which define the mathematical function that the neural network represents.

The raw training dataset 319n used for training may include noise and bad training images from the training database 144. Accordingly, to create a clean and filtered training dataset, the optional filter module 317 is configured to filter out unwanted data points from the raw training dataset 319n by developing smaller, less accurate systems based on patterns and metadata information.

During the training process, the facial landmark detection model trainer 325 is presented with input data and labels of actual values, and the optimization objective, which aims to minimize the difference between the actual value and the predicted value, is calculated. The optimization algorithm updates the parameters of the facial landmark detection model trainer 325 to reduce the value of the objective. This process is repeated for several iterations until the parameters do not change anymore. This process is repeated for various combinations of hyperparameters, and the model with the smallest label prediction error is selected as the final model.

When a new model (e.g., the pre-trained facial landmark detection MLM 126) is created, and a new process for filtering and automated labeling is established, it is added to the MLM database 146 in the MLM training module 124a. This enables the new model to be part of the closed-loop model update process. Optionally, at regular intervals, data which is continuously collected can be filtered, labeled, and used to update old models by an optional filtering ML module 329. In some examples, the optional filtering ML module 329 is a neural network. In some examples, the optional filtering ML module 329 is a mathematical model. This approach may capture changes in the data over time.

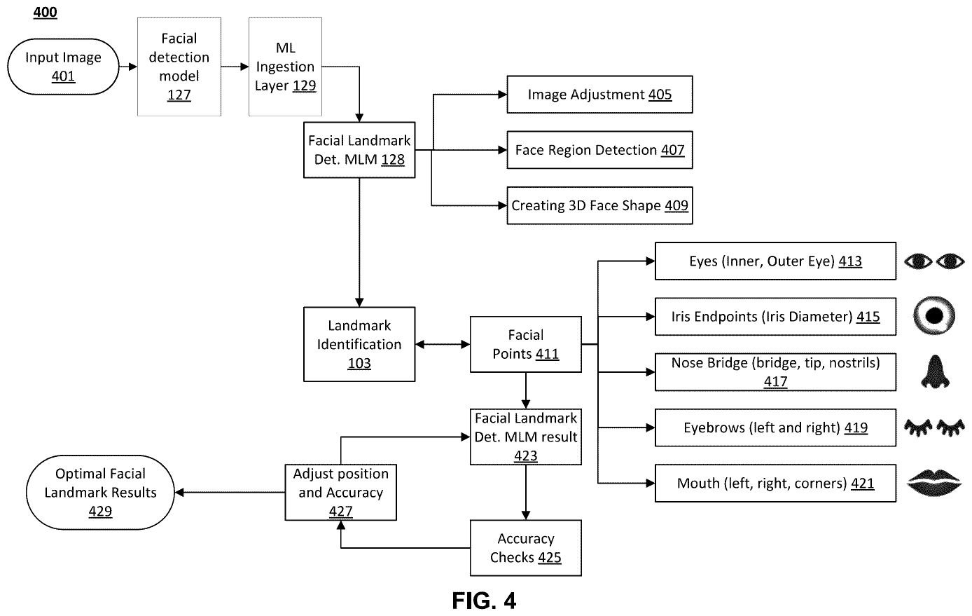

FIG. 4 is an example flowchart for calculating distances of facial landmark estimates for each user according to aspects of the present disclosure. In various implementations, the method 400 is performed by a device with one or more processors and non-transitory memory that performs intent prediction. In some implementations, the method 400 is performed by processing logic, including hardware, firmware, software, or a combination thereof. In some implementations, the method 400 is performed by a processor executing code stored in a non-transitory computer-readable medium (e.g., a memory). The method 400 describes a method of detecting facial landmark estimates from video frames capturing a face of a user.

As described in more detail in FIG. 1, the method 400 begins by inputting an image 401 (e.g., a single frame from a video) of a user's face into a facial detection model 127. A ML ingestion layer 129 may process the outputs of the facial detection/landmark model before sending a refined dataset of landmark output measurements to the facial landmark detection MLM 126. Upon receiving the input, the facial landmark detection MLM 126 performs a series of sequential operations to prepare and analyze the image 401. First, it generates an image adjustment 405 to optimize visual quality and alignment, enhancing conditions such as brightness, contrast, and orientation. Next, it performs face region detection 407 to accurately isolate the facial area from the background. Following this, the system constructs a 3D face shape 409 based on depth cues and facial geometry, providing a more accurate representation of facial contours. Finally, the model executes landmark identification 103, detecting and mapping key facial points that are critical for subsequent measurement and analysis tasks.

The landmark identification 103 process generates a comprehensive set of facial points 411 by analyzing the input image 401 and mapping key anatomical features with high precision. Using a deep learning model trained on diverse facial datasets, the system identifies and localizes critical facial regions, including at least the eyes 413, iris endpoints 415, nose bridge 417, eyebrows 419, and mouth 421. Each of these facial points is assigned a specific coordinate within the image, representing its exact location. This detailed mapping allows the method 400 to capture the user's unique facial geometry, enabling accurate measurements and feature recognition. These facial points serve as the foundational inputs for downstream calculations and personalized recommendations, such as eyewear fitting.

The facial landmark determination process includes a feedback loop in which the facial landmark detection MLM result 423 undergoes an accuracy check 425 to validate the precision of the detected facial landmark estimates. During this step, the method 400 evaluates factors such as landmark consistency across frames, alignment with expected anatomical ratios, and detection confidence scores. If any discrepancies or deviations are identified, the method 400 triggers an adjusting position and accuracy phase 427. This phase may involve recalibrating the user's position, prompting for improved lighting or focus, or correcting landmark placements using secondary validation methods. The updated image or frame is then reprocessed by the facial landmark detection MLM 126 to refine the landmark detection 429. This loop continues iteratively until the method 400 achieves optimal facial landmark results with sub-millimeter accuracy, ensuring reliable data for further measurement and analysis.

It is understood that the method illustrated by FIG. 4 is exemplary in nature and that the steps described herein may be combined or modified to generate alternative embodiments.

FIG. 5 is a block diagram illustrating a facial landmark mapping system for accurately detecting facial landmark estimates according to aspects of the present disclosure. Specifically, FIG. 5 provides a detailed visualization of a facial landmark mapping system 500 for measuring analyzing facial features for applications such as generating prescriptions and/or custom eyewear fitting.

In some aspects, the disclosed facial landmark mapping system 500 utilizes at least one MLM (e.g., the facial landmark detection MLM 126) to process individual image frames or a continuous stream of image data of a face 501 of a user. The facial landmark mapping system 500 is operative to generate output comprising 3D facial landmark estimates and blend shape coefficients, which quantitively represent facial expressions. These outputs may be employed to reconstruct detailed 3D facial surface geometry in real-time.

Specifically, the facial landmark detection MLM is configured to map a mesh grid 503 with black dot markers 505 that represent key facial landmark estimates (e.g., specific anatomical points used by the system in facial analysis) over a frame showing a face 501 of a user. The mesh grid 503 symbolizes the 468 3D landmarks that the facial landmark detection MLM detects in real-time. These landmarks are crucial for high-precision facial analysis and are categorized across various facial regions to support downstream applications such as calculating a distance between facial landmark estimates, generating prescription-related measurements, and/or recommending eyewear.

Each of the labeled facial features (e.g., eyebrows 507, eyes 509, temple to temple width 511, iris endpoints 513, nose bridge 515, and/or IPD distance 517) correspond to specific landmark indices output by the facial landmark detection MLM. For example, the eyebrows 507 (left and right) are tracked using indices, which are used to align the upper portion of eyeglass frames. The eyes 509 (left and right) are identified using landmarks around the eye contours, which are used to enable accurate measurement of eye width and orientation. The iris endpoints 513 (left and right) are particularly important for determining iris diameter and aligning lenses. The facial landmark detection MLM may also identify key points along the nose bridge 515, which are used to ensure frames sit securely on the nose. The IPD can be calculated by measuring the distance between iris centers, while temple to temple width 511 is approximated by the horizontal span between landmarks. It should be noted that although the present disclosure lists specific indices, the indices are an approximation and may also be used in combination with other indices to improve measurement accuracy.

Together, these measurements feed into a multi-step process: beginning with adjusting the position and accuracy of the landmarks (e.g., initializing detection and validating confidence scores), where landmarks are fine-tuned for precision; followed by generating optimal facial landmark results, which generates a data set of facial dimensions; and extracting a set of facial landmark estimates, where the system identifies anatomical features of the face 501 of the user.

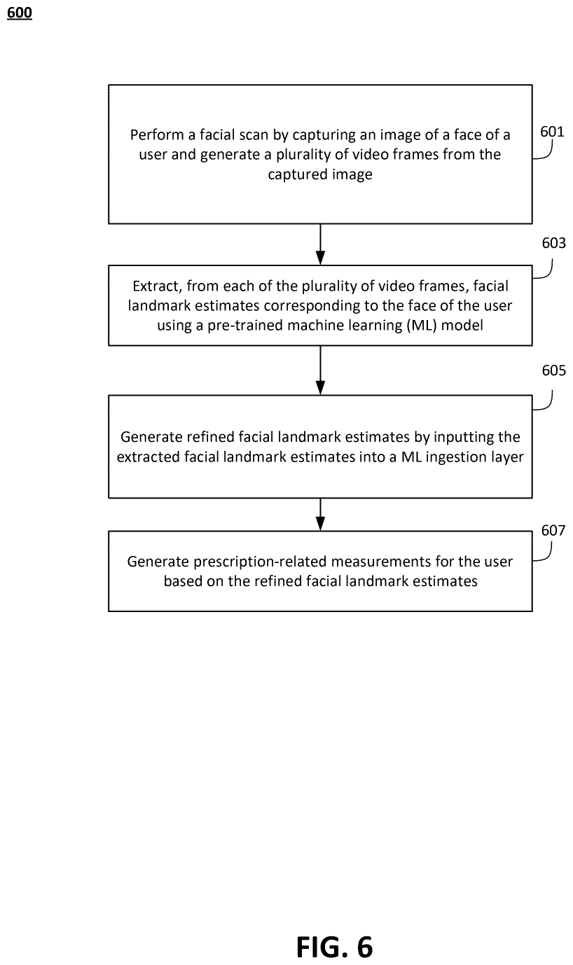

FIG. 6 is an example method for enhancing facial landmark accuracy according to an aspect of the present disclosure. In various implementations, the method 600 is performed by a device with one or more processors and non-transitory memory that performs intent prediction. In some implementations, the method 600 is performed by processing logic, including hardware, firmware, software, or a combination thereof. In some implementations, the method 600 is performed by a processor executing code stored in a non-transitory computer-readable medium (e.g., a memory). The method 600 describes a method of enhancing facial landmark accuracy via machine learning-based ingestion layer according to aspects of the present disclosure.

At 601, the method 600 may include perform a facial scan by capturing an image of a face of a user and generate a plurality of video frames from the captured image. This video feed provides the necessary input for continuous facial landmark tracking, allowing the system to identify and monitor key facial features in real time. The process serves as the foundation for subsequent analysis or interaction, ensuring that facial movements and expressions are consistently and accurately tracked throughout the session.

For example, the method 600 may include initializing a facial scan of a user by acquiring, via one or more image sensors, a set of video data capturing at least a portion of the user's face for the purpose of enabling continuous facial landmark tracking. The acquired video data may comprise a sequence of image frames recorded over time, wherein each frame includes visual information corresponding to the facial region of the user. The continuous feed of image data may be processed in real time or near real time to detect and track a plurality of facial landmark estimates, including but not limited to, the corners of the eyes, mouth, nose tip, eyebrows, and jawline. This initialization step establishes the necessary input for ongoing facial analysis by providing dynamic, temporally-consistent visual input, thereby facilitating accurate identification and monitoring of user facial expressions and movements throughout the duration of the session. Such continuous landmark tracking may be used to support downstream applications including user authentication, expression recognition, head pose estimation, or gesture-based interaction.

In some aspects, obtaining the set of video data of the face of the user further comprises: tracking face positioning of the user by applying a bounding box to ensure that the face of the user is centered, and, based on a determination that a percentage of the face of the user is outside of the bounding box, pausing the facial scan and prompting the user to re-center. Specifically, the method 600 further enhances the video data capture process by actively tracking the positioning of the user's face. This is achieved by applying a bounding box around the detected face to ensure it remains centered within the frame. The system continuously monitors the alignment of the face, and if it determines that a significant portion of the face has moved outside the bounding box-indicating improper positioning-it temporarily pauses the facial scan. At this point, the user is prompted to adjust their position and re-center their face within the frame to resume the scanning process. This ensures consistent data quality and accuracy in subsequent facial analysis.

In some aspects, obtaining the set of video data of the face of the user further comprises: evaluating brightness across a facial region of the user, and, based on a determination that a brightness across the facial region of the user is below a first threshold or above a second threshold, pausing the scan and prompting the user to adjust lighting. For example, the method 600 may include includes assessing the lighting conditions on the user's face during the video data capture process. Specifically, the system evaluates the brightness levels across the facial region to ensure proper illumination. If the detected brightness falls below a predefined lower threshold or exceeds an upper threshold—indicating that the lighting is either too dim or overly bright—the system pauses the facial scan. It then prompts the user to adjust their environment or lighting setup to achieve optimal brightness. This step helps maintain the quality and reliability of the facial data by ensuring that key features are clearly visible for accurate landmark detection and analysis.

At 603, the method 600 may include extracting, from each of the plurality of video frames, facial landmark estimates corresponding to the face of the user using a pre-trained ML model configured to identify anatomical features of the face. The extracted facial landmark estimates may include at least one of a pupil estimate, an iris edge estimates, a 68-point model, or temple-to-temple width. The set of facial landmark estimates may include at least eye measurements, pupil measurements, and temple to temple width. For example, the method 600 involves extracting facial landmark estimates from each frame of the captured video data using a pre-trained MLM. This model is specifically designed to recognize and identify anatomical features of the user's face with high accuracy. As the video is processed frame by frame, the system detects a consistent set of facial landmark estimates, which typically includes the eyes, pupils, and temple to temple width. These landmarks serve as critical reference points for further analysis, enabling applications such as facial recognition, alignment, and custom fitting tasks like eyewear measurement or augmented reality overlay.

At 605, the method 600 may include generating refined facial landmark estimates by inputting the extracted facial landmark estimates into a ML ingestion layer configured to perform at least one of: filtering noise, correcting drift, or improving reliability of the extracted facial landmarks. For example, the ML ingestion layer may be configured to perform one or more refinement operations on the raw landmark data, such as filtering out measurement noise, correcting for temporal or spatial drift, and enhancing the overall reliability and consistency of the landmark output. The input facial landmark estimates may include, but are not limited to, coordinates corresponding to the pupils, iris edge, a standardized 68-point facial landmark model, and measurements indicative of temple-to-temple facial width. The ML ingestion layer may utilize one or more trained models, statistical filters, or neural network architectures to identify inconsistencies, anomalies, or outliers in the landmark data and apply corrective adjustments in real time or in batch. The resulting refined dataset may exhibit improved precision and temporal stability, thereby serving as a more reliable foundation for subsequent facial analysis tasks, such as expression tracking, biometric identification, or user interaction control.

In some aspects, improving the reliability of the extracted facial landmark estimates further includes: tracking the stability of a plurality of the extracted facial landmark estimates, and discarding the extracted facial landmark estimates that have a stability that does not conform to a predetermined stability threshold. In some aspects, generating the refined dataset of landmark output measurements may further include performing temporal analysis to track facial landmark stability over a sequence of image frames. This may involve monitoring the position and movement of each landmark over time to assess consistency and detect anomalies. The system may analyze whether deviations in landmark positions represent valid anatomical movements or constitute errant data points. To this end, the system may determine whether identified outliers conform to predicted anatomical movement patterns based on learned or predefined biomechanical models of facial motion. Outliers that do not conform to such predicted anatomical behavior (e.g., sudden, non-physiological shifts in landmark position or inconsistent motion relative to surrounding features) may be classified as erroneous and discarded from the dataset. This selective exclusion process enhances the integrity and physiological plausibility of the refined landmark output, ensuring that only stable and anatomically coherent data is retained for downstream facial analysis operations.

In some aspects, the method 600 may include extracting facial landmark estimate data measured in a pixel-space from the refined facial landmark estimates. The facial landmark estimate data may include at least one of: iris boundary, centers of pupils, midpoints between an upper and lower eyelid landmark, a 68-point model, or the widest part of the face. For example, the extraction process may involve isolating coordinate values and spatial relationships of key facial features as represented in the pixel-space of the image or video frames. The facial landmark data may include, but is not limited to: the boundary contours of the irises; the geometric centers of the pupils; the midpoints between corresponding upper and lower eyelid landmarks; a standardized 68-point facial landmark configuration encompassing critical facial regions such as eyes, nose, mouth, and jawline; and measurements corresponding to the widest horizontal span of the face, such as temple-to-temple distance. By referencing these landmarks in pixel-space, the system preserves high-resolution positional accuracy suitable for precise facial analysis, enabling downstream tasks such as gaze tracking, facial geometry modeling, expression recognition, and augmented reality alignment.

In some aspects, extracting the facial landmark data may further include: selecting pairs of facial landmark estimates from the refined dataset of landmark output measurements and the set of video data using the pre-trained machine learning model, and calculating a Euclidean distance between each selected pair of facial landmark estimates. For example, the pre-trained model may be configured to identify relevant landmark pairs based on anatomical significance, spatial arrangement, or task-specific criteria. Once selected, the method 600 may include calculating a Euclidean distance between each pair of facial landmark estimates, wherein the distance is computed based on the pixel-space coordinates of the respective landmarks within the image or video frames. These distance measurements may be used to assess relative positioning, monitor facial dynamics, or detect changes in facial expressions or orientation. By leveraging the pre-trained model to guide landmark pair selection, the system ensures that distance calculations are focused on semantically meaningful relationships, thereby enhancing the precision and contextual relevance of the extracted facial landmark data.

In some aspects, the pairs of facial landmark estimates comprise at least one of: center of left pupil and center of right pupil for measuring IPD; a first outer iris edge and a second outer iris edge for measuring iris width; a first nose bridge point and a second nose bridge point; and a mid-point of an ear to an outer corner of an eye for measuring temple arm length. For example, the pairs of facial landmark estimates may comprise at least one of the following: a pair including the center of the left pupil and the center of the right pupil, configured for measuring IPD; a pair including a first outer iris edge and a second outer iris edge, configured for determining iris width; a pair comprising a first nose bridge point and a second nose bridge point, for characterizing nasal geometry; and a pair including a midpoint of an ear and an outer corner of an eye, configured for estimating temple arm length. These specific landmark pairings may be selected based on their anatomical relevance to biometric measurement, eyewear fitting, or facial geometry analysis. By computing distances between these landmark pairs, the system may derive personalized spatial metrics that inform downstream applications such as optical device customization, user profiling, and real-time fit estimation.

At 607, the method 600 may include generate prescription-related measurements for the user based on the refined facial landmark estimates. The prescription-related measurements may include at least one of a temple to temple width, temple arm length, or a nose bridge width. This scaling factor enables conversion of pixel-space coordinates into real-world dimensional units (e.g., millimeters), thereby allowing precise estimation of the user's physical facial features. The resulting facial fit measurement may include, but is not limited to: an iris width measurement indicating the physical diameter of the iris; an IPD measurement corresponding to the distance between the centers of the user's pupils; a nose bridge width distance determined between selected nasal bridge landmarks; and a temple-to-temple distance indicating the lateral span of the user's face. These real-world measurements offer significant benefits, including improved accuracy in virtual or augmented reality headset fitting, personalized eyewear sizing, ergonomic customization of head-mounted devices, and enhanced biometric verification. By transforming image-based landmark data into precise physical metrics, the system facilitates user-specific product recommendations and optimized user experience across a range of wearable and vision-based technologies.

In some aspects, the method 600 may further include generating the plurality of video frames in pixel space, and generating the prescription-related measurements based further on the refined facial landmark estimates in real-world measurements by applying a scaling factor to the extracted facial landmark estimates or to the refined facial landmark estimates. In some aspects, determining the scaling factor includes: determining the scaling factor by (W/iris_width_pixels)+(WIPD/pupil_distance_pixel)/2. W corresponds to a predefined iris width constant representing the average or known physical diameter of a human iris, and WIPD corresponds to a predefined IPD constant representing the average or known physical distance between the centers of the pupils. iris_width_pixels denotes the pixel-space distance measured between opposing outer edges of the iris, and pupil_distance_pixels denotes the pixel-space distance between the centers of the left and right pupils. By averaging the scaling factors derived from both the iris and the interpupillary measurements, the system achieves a more robust and accurate estimation of the real-world scaling factor, which can then be applied to convert additional pixel-based facial measurements into corresponding real-world units. This dual-reference approach helps mitigate individual measurement errors and improves overall precision of facial dimension estimation.

In some aspects, the method 600 may include generating a reliability score or an accuracy score for each facial landmark from the refined dataset of landmark output measurements. This process may involve analyzing each landmark's positional consistency, temporal stability, and adherence to expected anatomical relationships across sequential frames or data samples. The system may utilize statistical models, confidence outputs from machine learning inference, or comparison with established biomechanical norms to assign a reliability score indicating the confidence level associated with each landmark's measurement. Similarly, an accuracy score may be generated by comparing the measured position of a landmark to a predicted or calibrated reference point, reflecting the landmark's deviation from an expected location. These scores may be computed independently or in combination, and may be dynamically updated as new data is captured. The inclusion of such scoring metrics provides several benefits, including improved robustness in facial analysis by allowing downstream systems to weight or discard low-confidence data, increased reliability in applications requiring precise facial geometry, and enhanced transparency in system performance, enabling real-time diagnostics or adaptive system behavior based on landmark quality. By quantifying the quality of each facial landmark, the system supports more accurate and stable outcomes across diverse real-world conditions.

In some aspects, the machine learning model is trained using a supervised learning approach on a training dataset comprising facial images annotated with ground truth anatomical features to predict one or more of eye-to-eye distance, iris width, inter-pupillary distance, temple to temple width, temple arm length, and nose bridge length. Each image in the training dataset may be labeled with precise measurements or positions of key anatomical landmarks, such as the eye centers, iris boundaries, nose bridge points, and facial contours. During the training phase, the model learns to associate pixel-space features and spatial relationships in the input images with these annotated ground truth values. The objective of the supervised learning process is to enable the model to accurately predict one or more anatomical dimensions from new, unseen facial images, including but not limited to: eye-to-eye distance, iris width, IPD, temple-to-temple width, temple arm length, and nose bridge length. This training approach allows the model to generalize across a diverse population of users and image conditions, improving prediction robustness under variations in lighting, pose, and facial geometry. The benefits of this method include high-precision estimation of facial dimensions in real time, scalability to large user populations, and adaptability to different hardware environments. Additionally, by leveraging ground truth anatomical data, the system ensures clinically or ergonomically relevant accuracy, which is critical for applications such as custom eyewear fitting, biometric authentication, and head-mounted display calibration.

In some aspects, the training dataset comprises at least one of: facial images annotated with a distance between an outer or inner corners of both eyes of a user; facial images annotated with left and right boundaries of each iris; facial images annotated with a pupil center for each eye; facial images annotated with landmarks at a widest visible part of the face; facial images annotated with a nasion point or a bottom of a nose bridge; and facial images annotated with a mid-point of an ear to an outer corner of the eye.

For example, the disclosed system utilizes the training dataset comprising a plurality of facial images, each annotated with one or more facial feature landmarks, wherein the annotations include at least one of the following: (i) a distance measurement between the outer canthi (outer corners) or inner canthi (inner corners) of both eyes of a subject depicted in the image, wherein said canthi are identified based on the intersection of the upper and lower eyelid contours, and the computed inter-canthus distance serves as a reference metric for determining image scale, normalizing face size, or calibrating interocular spatial relationships; (ii) positional boundaries of the left and right sides of each iris, the annotations comprising two or more coordinate points along a horizontal axis that bisects the iris at its widest visible portion, thereby enabling calculation of iris width, estimation of gaze direction, or refinement of eye-region segmentation in downstream processing; (iii) a centrally located pupil point for each eye, wherein the pupil center is annotated based on image intensity minima or geometric center detection within the iris region, and is configured to support pupil-tracking algorithms, gaze estimation, and individualized biometric calibration; (iv) a set of facial landmark estimates corresponding to the widest visible part of the face, the landmarks being disposed at lateral extremities such as the outermost cheekbones or jawline edges as determined under frontal or near-frontal imaging conditions, such landmarks providing a basis for determining facial aspect ratios, orientation normalization, and dynamic face model fitting; (v) a nasion point, defined as the midpoint of the intersection between the frontal bone and the two nasal bones at the bridge of the nose, or alternatively a bottom-of-nose-bridge point corresponding to the lowest visible inflection point on the nasal dorsum, each of which may function as a critical craniofacial reference marker for establishing midline symmetry, performing depth inference, or initializing three-dimensional head pose estimation models; and (vi) a reference point located at a geometric midpoint between a visible portion of the helix or tragus of the ear and the outer canthus of the adjacent eye, the computed midpoint serving as a baseline vector component for determining lateral facial dimensions, angular head rotation, or for anchoring virtual overlays in augmented reality (AR) and virtual reality (VR) applications. The annotations may be generated manually by human annotators, semi-automatically using landmark detection algorithms, or fully automatically via prior-trained neural networks, and are associated with their respective facial images through structured data formats such as JSON, XML, or integrated facial landmark map encodings. During training, the annotated images are input to one or more machine learning models (e.g., CNNs, transformer-based vision architectures, or hybrid regression networks) configured to learn spatial relationships, topological facial feature patterns, and predictive gaze or head pose parameters. The system may further incorporate preprocessing steps including histogram equalization, affine normalization, and facial alignment to standardize image input prior to training. By leveraging this annotated dataset, the model learns to infer corresponding landmarks and distances in unannotated input images with high spatial accuracy and robustness across diverse lighting conditions, occlusions, and facial orientations, thereby enabling applications including facial measurements but not limited to biometric authentication, facial recognition, gaze tracking, emotion detection, and AR/VR headset calibration.

It is understood that the method illustrated by FIG. 6 is exemplary in nature and that the steps described herein may be combined or modified to generate alternative embodiments.

FIG. 7 is a block diagram illustrating a computer system 20 on which aspects of systems and methods machine learning (ML)-based analysis of determining prescription-related facial measurements for fitting eyewear may be implemented. The computer system 20 can be in the form of multiple computing devices, or in the form of a single computing device, for example, a desktop computer, a notebook computer, a laptop computer, a mobile computing device, a smart phone, a tablet computer, a server, a mainframe, an embedded device, and other forms of computing devices.

As shown, the computer system 20 includes a central processing unit (CPU) 21, a system memory 22, and a system bus 23 connecting the various system components, including the memory associated with the central processing unit 21. The system bus 23 may comprise a bus memory or bus memory controller, a peripheral bus, and a local bus that is able to interact with any other bus architecture. Examples of the buses may include PCI, ISA, PCI-Express, HyperTransport™, InfiniBand™, Serial ATA, 12C, and other suitable interconnects. The central processing unit 21 (also referred to as a processor) can include a single or multiple sets of processors having single or multiple cores. The processor 21 may execute one or more computer-executable code implementing the techniques of the present disclosure. For example, any of commands/steps discussed in FIGS. 1-6 may be performed by processor 21. The system memory 22 may be any memory for storing data used herein and/or computer programs that are executable by the processor 21. The system memory 22 may include volatile memory such as a random access memory (RAM) 25 and non-volatile memory such as a read only memory (ROM) 24, flash memory, etc., or any combination thereof. The basic input/output system (BIOS) 26 may store the basic procedures for transfer of information between elements of the computer system 20, such as those at the time of loading the operating system with the use of the ROM 24.

The computer system 20 may include one or more storage devices such as one or more removable storage devices 27, one or more non-removable storage devices 28, or a combination thereof. The one or more removable storage devices 27 and non-removable storage devices 28 are connected to the system bus 23 via a storage interface 32. In an aspect, the storage devices and the corresponding computer-readable storage media are power-independent modules for the storage of computer instructions, data structures, program modules, and other data of the computer system 20. The system memory 22, removable storage devices 27, and non-removable storage devices 28 may use a variety of computer-readable storage media. Examples of computer-readable storage media include machine memory such as cache, SRAM, DRAM, zero capacitor RAM, twin transistor RAM, eDRAM, EDO RAM, DDR RAM, EEPROM, NRAM, RRAM, SONOS, PRAM; flash memory or other memory technology such as in solid state drives (SSDs) or flash drives; magnetic cassettes, magnetic tape, and magnetic disk storage such as in hard disk drives or floppy disks; optical storage such as in compact disks (CD-ROM) or digital versatile disks (DVDs); and any other medium which may be used to store the desired data and which can be accessed by the computer system 20.

The system memory 22, removable storage devices 27, and non-removable storage devices 28 of the computer system 20 may be used to store an operating system 35, additional program applications 37, other program modules 38, and program data 39. The computer system 20 may include a peripheral interface 46 for communicating data from input devices 40, such as a keyboard, mouse, stylus, game controller, voice input device, touch input device, or other peripheral devices, such as a printer or scanner via one or more I/O ports, such as a serial port, a parallel port, a universal serial bus (USB), or other peripheral interface. A display device 47 such as one or more monitors, projectors, or integrated display, may also be connected to the system bus 23 across an output interface 48, such as a video adapter. In addition to the display devices 47, the computer system 20 may be equipped with other peripheral output devices (not shown), such as loudspeakers and other audiovisual devices.

The computer system 20 may operate in a network environment, using a network connection to one or more remote computers 49. The remote computer (or computers) 49 may be local computer workstations or servers comprising most or all of the aforementioned elements in describing the nature of a computer system 20. Other devices may also be present in the computer network, such as, but not limited to, routers, network stations, peer devices or other network nodes. The computer system 20 may include one or more network interfaces 51 or network adapters for communicating with the remote computers 49 via one or more networks such as a local-area computer network (LAN) 50, a wide-area computer network (WAN), an intranet, and the Internet. Examples of the network interface 51 may include an Ethernet interface, a Frame Relay interface, SONET interface, and wireless interfaces.

Aspects of the present disclosure may be a system, a method, and/or a computer program product. The computer program product may include a computer readable storage medium (or media) having computer readable program instructions thereon for causing a processor to carry out aspects of the present disclosure.

As used herein, the term “processor” is intended to be interpreted broadly and may refer to any hardware, firmware, software, or combination thereof configured to carry out one or more of the described operations, including, without limitation, signal processing, artificial intelligence functions, machine learning, deep learning, neural network inference, training, feature extraction, pattern recognition, and recommendation generation. A processor may be implemented using, without limitation, a general-purpose processor (GPP) such as a microprocessor or central processing unit (CPU), a digital signal processor (DSP), an application-specific integrated circuit (ASIC), a field-programmable gate array (FPGA) or other programmable logic device (PLD), a neural network accelerator, tensor processing unit (TPU), or other AI-specific processing unit, a graphics processing unit (GPU) configured for parallel or matrix computations, a microcontroller, a state machine, discrete gate or transistor-level logic, or any combination thereof, including configurations in which functions are split or shared among multiple components. The processor may be embodied as a single computing device or distributed across multiple devices or entities operating together. For example, processing may be performed by a combination of one or more microprocessors, one or more DSPs, and one or more neural network engines operating cooperatively or in parallel. In certain embodiments, the processor may be implemented in a heterogeneous system-on-chip (SoC) including CPU, DSP, GPU, and Al cores. The term “processor” should be understood to encompass multi-core, multi-threaded, or virtual processing environments, and may operate in conjunction with or under the control of software and/or firmware instructions stored in a non-transitory computer-readable medium. Unless expressly limited, no specific architecture, instruction set, or processing model is required. All such configurations, whether now known or later developed, that are capable of performing the described functionality are within the scope of the term “processor” as used herein.

A non-transitory computer-readable medium may store instructions for execution by one or more processors to perform any of the methods, processes, algorithms, or functions described herein. The non-transitory computer-readable medium described herein may be located on, off, or distributed with respect to the processor or processors that execute the instructions stored thereon. For example, the medium may include on-chip memory components, such as registers, level 1 (L1) or level 2 (L2) caches, or other memory blocks physically integrated with the processor (e.g., in a system-on-chip (SoC) or multi-core architecture). Alternatively or additionally, the medium may include off-chip or external memory components, such as random access memory (RAM), read-only memory (ROM), flash memory, solid-state drives (SSDs), hard drives, or networked or remote storage systems, including cloud-based or distributed storage architectures. The non-transitory computer-readable medium may also be logically or physically distributed across multiple memory devices or components. For example, a portion of the instructions may be stored in cache on the processor, while other portions reside in external memory modules or remote memory systems accessible via a bus or network interface. In some embodiments, the medium may include storage that is shared across multiple processors (e.g., shared memory in a multi-core environment), or distributed between processors and peripheral components, such as co-processors, accelerators, or I/O controllers. The computer-readable medium may further store data sets, machine learning models, parameters, or other information used in connection with artificial intelligence-based image processing, facial analysis, or recommendation generation. Accordingly, the computer-readable medium may be part of the processor, reside external to the processor, or be distributed across both, including within or across one or more computing entities, such as a combination of CPUs, DSPs, GPUs, neural network accelerators, and associated memory hierarchies as previously defined herein. The architecture may be implemented in a centralized, decentralized, or distributed computing environment, without limitation, and the term “non-transitory computer-readable medium” should be interpreted broadly to encompass all such configurations.

The computer readable storage medium can be a tangible device that can retain and store program code in the form of instructions or data structures that can be accessed by a processor of a computing device, such as the computing system 20. The computer readable storage medium may be an electronic storage device, a magnetic storage device, an optical storage device, an electromagnetic storage device, a semiconductor storage device, or any suitable combination thereof. By way of example, such computer-readable storage medium can comprise a random access memory (RAM), a read-only memory (ROM), EEPROM, a portable compact disc read-only memory (CD-ROM), a digital versatile disk (DVD), flash memory, a hard disk, a portable computer diskette, a memory stick, a floppy disk, or even a mechanically encoded device such as punch-cards or raised structures in a groove having instructions recorded thereon. As used herein, a computer readable storage medium is not to be construed as being transitory signals per se, such as radio waves or other freely propagating electromagnetic waves, electromagnetic waves propagating through a waveguide or transmission media, or electrical signals transmitted through a wire.

Computer readable program instructions described herein can be downloaded to respective computing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network interface in each computing device receives computer readable program instructions from the network and forwards the computer readable program instructions for storage in a computer readable storage medium within the respective computing device.

Computer readable program instructions for carrying out operations of the present disclosure may be assembly instructions, instruction-set-architecture (ISA) instructions, machine instructions, machine dependent instructions, microcode, firmware instructions, state-setting data, or either source code or object code written in any combination of one or more programming languages, including an object oriented programming language, and conventional procedural programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a LAN or WAN, or the connection may be made to an external computer (for example, through the Internet). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present disclosure.

In various aspects, the systems and methods described in the present disclosure can be addressed in terms of modules. The term “module” as used herein refers to a real-world device, component, or arrangement of components implemented using hardware, such as by an application specific integrated circuit (ASIC) or FPGA, for example, or as a combination of hardware and software, such as by a microprocessor system and a set of instructions to implement the module's functionality, which (while being executed) transform the microprocessor system into a special-purpose device. A module may also be implemented as a combination of the two, with certain functions facilitated by hardware alone, and other functions facilitated by a combination of hardware and software. In certain implementations, at least a portion, and in some cases, all, of a module may be executed on the processor of a computer system. Accordingly, each module may be realized in a variety of suitable configurations and should not be limited to any particular implementation exemplified herein.

As used herein, the term “image sensor” refers to any device, circuit, system, or component that is capable of capturing, detecting, acquiring, or converting an optical image, light signal, or visual representation into an electrical signal or digital data. The image sensor may be implemented using charge-coupled device (CCD) technology, complementary metal-oxide-semiconductor (CMOS) technology, active pixel sensors (APS), photodiode arrays, avalanche photodiodes, quantum dot-based sensors, organic photodetectors, infrared (IR) sensors, ultraviolet (UV) sensors, time-of-flight (ToF) sensors, lidar-based sensors, thermal imaging sensors, or any other technology now known or later developed that is capable of capturing an image or light information. The term “image sensor” also includes, without limitation, multi-modal sensors capable of capturing data across multiple spectral bands (e.g., visible, infrared, ultraviolet), depth sensors, 3D imaging sensors, structured light sensors, stereo cameras, and any sensor capable of acquiring biometric or anthropometric data relevant to facial analysis. In some embodiments, the image sensor may include or be associated with optical elements such as lenses, shutters, filters, or microlens arrays; signal conditioning components such as analog-to-digital converters or amplifiers; and control logic, which may be implemented via firmware, software, or hardware, to facilitate acquisition and processing of image data. The image sensor may operate under the control of a processor (as defined herein) and may be locally or remotely situated relative to such processor. The image sensor may be implemented as a single unit, a distributed array, or a plurality of cooperating sensors.

As used herein, the term “display” refers to any device, component, system, or interface configured to present visual information to a user or viewer, including but not limited to images, graphics, text, video, icons, user interface elements, or any other visually perceptible data. The display may include, without limitation, a liquid crystal display (LCD), light-emitting diode (LED) display, organic light-emitting diode (OLED) display, quantum-dot display (QLED or QD-OLED), plasma display, electrophoretic display (e.g., e-ink), microLED display, projection-based display, laser-based display, holographic display, head-up display (HUD), retinal projection display, 3D display, volumetric display, augmented reality (AR) or virtual reality (VR) head-mounted display, or any other display technology now known or later developed. The term “display” further includes any device or system capable of presenting augmented reality (AR) or virtual reality (VR) content, including overlays of virtual eyeglass frames on a user's face, or otherwise facilitating interactive visualization of recommended products. As used herein, the term “display” encompasses single-screen displays, multi-screen displays, curved or flexible displays, transparent displays, and modular display systems, as well as any associated control electronics, display drivers, timing controllers, graphics processing units (GPUs), backlight units, or optical components necessary to support image rendering and visibility.

In the interest of clarity, not all of the routine features of the aspects are disclosed herein. It would be appreciated that in the development of any actual implementation of the present disclosure, numerous implementation-specific decisions must be made in order to achieve the developer's specific goals, and these specific goals will vary for different implementations and different developers. It is understood that such a development effort might be complex and time-consuming but would nevertheless be a routine undertaking of engineering for those of ordinary skill in the art, having the benefit of this disclosure.

Furthermore, it is to be understood that the phraseology or terminology used herein is for the purpose of description and not of restriction, such that the terminology or phraseology of the present specification is to be interpreted by the skilled in the art in light of the teachings and guidance presented herein, in combination with the knowledge of those skilled in the relevant art(s). Moreover, it is not intended for any term in the specification or claims to be ascribed an uncommon or special meaning unless explicitly set forth as such.

The various aspects disclosed herein encompass present and future known equivalents to the known modules referred to herein by way of illustration. Moreover, while aspects and applications have been shown and described, it would be apparent to those skilled in the art having the benefit of this disclosure that many more modifications than mentioned above are possible without departing from the inventive concepts disclosed herein.

What is claimed is:

1. A system for enhancing facial landmark accuracy, comprising:
an image sensor configured to perform a facial scan by capturing an image of a face of a user and generate a plurality of video frames from the captured image; and
a processor coupled to the image sensor, the processor being configured to:
extract, from each of the plurality of video frames, facial landmark estimates corresponding to the face of the user using a pre-trained machine learning (ML) model configured to identify anatomical features of the face, wherein the extracted facial landmark estimates comprise at least one of a pupil estimate, an iris edge estimates, a 68-point model, or temple-to-temple width;
generate refined facial landmark estimates by inputting the extracted facial landmark estimates into a ML ingestion layer configured to perform at least one of: filtering noise, correcting drift, or improving reliability; and
generate prescription-related measurements for the user based on the refined facial landmark estimates, wherein the prescription-related measurements comprises at least one of a temple to temple width, temple arm length, or a nose bridge width.

2. The system of claim 1, wherein the image sensor is configured to:
generate the plurality of video frames in pixel space,
wherein the processor is further configured to:
generate the prescription-related measurements based further on the refined facial landmark estimates in real-world measurements by applying a scaling factor to the extracted facial landmark estimates or to the refined facial landmark estimates.

3. The system of claim 2, wherein the processor is further configured to: determine the scaling factor by (W/iris_width_pixels)+(WIPD/pupil_distance_pixel)/2, wherein W corresponds to an iris width constant and WIPD corresponds to an IPD distance.

4. The system of claim 1, wherein the processor is further configured to improve the reliability of the extracted facial landmark estimates by:
tracking the stability of a plurality of the extracted facial landmark estimates, and
discarding the extracted facial landmark estimates that have a stability that does not conform to a predetermined stability threshold.

5. The system of claim 1, wherein the machine learning model is trained using a supervised learning approach on a training dataset comprising facial images annotated with ground truth anatomical features.

6. The system of claim 5, wherein the training dataset comprises at least one of:
facial images annotated with a distance between an outer or inner corners of both eyes;
facial images annotated with left and right boundaries of each iris;
facial images annotated with a pupil center for each eye;
facial images annotated with landmarks at a widest visible part of the face;
facial images annotated with a nasion point or a bottom of a nose bridge; and
facial images annotated with a mid-point of an ear to an outer corner of the eye.

7. A method for enhancing facial landmark accuracy, comprising:
performing a facial scan by capturing an image of a face of a user and generating a plurality of video frames from the captured image;
extracting, from each of the plurality of video frames, facial landmark estimates corresponding to the face of the user using a pre-trained machine learning (ML) model configured to identify anatomical features of the face, wherein the extracted facial landmark estimates comprise at least one of a pupil estimate, an iris edge estimates, a 68-point model, or temple-to-temple width;
generating refined facial landmark estimates by inputting the extracted facial landmark estimates into a ML ingestion layer configured to perform at least one of: filtering noise, correcting drift, or improving reliability; and
generating prescription-related measurements for the user based on the refined facial landmark estimates, wherein the prescription-related measurements comprises at least one of a temple to temple width, temple arm length, or a nose bridge width.

8. The method of claim 7, further comprising:
generating the plurality of video frames in pixel space; and
generating the prescription-related measurements based further on the refined facial landmark estimates in real-world measurements by applying a scaling factor to the extracted facial landmark estimates or to the refined facial landmark estimates.

9. The method of claim 8, further comprising:
determining the scaling factor by (W/iris_width_pixels)+(WIPD/pupil_distance_pixel)/2, wherein W corresponds to an iris width constant and WIPD corresponds to an IPD distance.

10. The method of claim 7, further comprising:
tracking the stability of a plurality of the extracted facial landmark estimates; and
discarding the extracted facial landmark estimates that have a stability that does not conform to a predetermined stability threshold.

11. The method of claim 7, wherein the machine learning model is trained using a supervised learning approach on a training dataset comprising facial images annotated with ground truth anatomical features.

12. The method of claim 11, wherein the training dataset comprises at least one of:
facial images annotated with a distance between an outer or inner corners of both eyes;
facial images annotated with left and right boundaries of each iris;
facial images annotated with a pupil center for each eye;
facial images annotated with landmarks at a widest visible part of the face;
facial images annotated with a nasion point or a bottom of a nose bridge; and
facial images annotated with a mid-point of an ear to an outer corner of the eye.
